(19) United States
(12) Patent Application Publication
(10) Pub. No.: US 2018/0249966 A1
(43) Pub. Date: Sep. 6, 2018

(54) SYSTEMS AND METHODS TO TRACK AND MANAGE INDIVIDUALIZED REDOX HOMEOSTASIS

(71) Applicant: ORRECO LTD, Sligo (IE)

(72) Inventors: Nathan A. Lewis, Cheltenham (GB); John Newell, Galway (IE); Charles R. Pedlar, Hampton Hill (GB); Brian Moore, Sligo (IE)

(21) Appl. No.: 15/907,837

(22) Filed: Feb. 28, 2018

Related U.S. Application Data
(60) Provisional application No. 62/464,924, filed on Feb. 28, 2017.

(51) Int. Cl. A61B 5/00; G16H 50/30; G01N 33/52

(52) U.S. Cl. CPC A61B 5/7275; G01N 33/52; G16H 50/30

(57) ABSTRACT

Systems and methods to track and manage individualized redox homeostasis are described. Tracking individualized redox homeostasis detects an increased risk of injury and/or illness thereby allowing appropriate interventions to reduce this risk.

### CROSS-REFERENCE TO RELATED APPLICATION

This application claims priority to 62/464,924 filed on Feb. 28, 2017, which is incorporated herein by reference in its entirety as if fully set forth herein.

### FIELD OF THE DISCLOSURE

The present disclosure describes systems and methods to track and manage individualized redox homeostasis. Tracking individualized redox homeostasis can detect an increased risk of injury and/or illness thereby allowing appropriate interventions to reduce this risk.

### SUMMARY OF THE DISCLOSURE

One of the biggest challenges in maintenance and improvement of health is maintaining a balance (redox homeostasis) between pro- and anti-oxidants. Maintaining this balance is essential for muscle function and training adaptation. This is because significant deviations from redox homeostasis are associated with degradations in physical performance, illness, and fatigue.

The current disclosure provides systems and methods to reduce the risk of injury and/or illness and monitor wellness by testing regularly for significant deviations from an individual's redox homeostasis. In particular embodiments, reducing the risk of injury and/or illness prevents an injury and/or illness from occurring in an individual. In particular embodiments, reducing the risk of injury and/or illness reduces the severity of an injury or illness, should it occur. The systems and methods utilize point-of-care testing to generate serial measures from individuals. Data from the serial measures is used to create individualized baselines and thresholds so that a individualized risk indicator is generated. In particular embodiments, the systems and methods utilize a rules base to generate automatic alerts and recommendations based on the personalized and/or individualized baseline and trend indicators. By tracking and monitoring redox homeostasis in individuals and detecting detrimental alterations in redox homeostasis (ARH), interventions can be implemented to reduce the risk of injury and illness, maintain current health status and/or sustain wellness.

In particular embodiments, there are two marker values derived from a subject sample test: a pro-oxidant score and an anti-oxidant score. A critical difference value is calculated based on serial measurements from a number of subjects for both the pro-oxidant and anti-oxidant scores. The critical difference value incorporates sources of measurement error and biological variation in the calculations. In particular embodiments, those sources of error are combined to compute a %, referred to as the critical difference value. In particular embodiments, the critical difference value can then be combined with the (healthy subject) mean (derived from serial measurements on the individual subject), to establish the subject's individual critical difference threshold value. This creates an individual range where future healthy data generated would fit. The critical difference threshold can oscillate as more “healthy” data is collected on the subject; thus the critical difference threshold can be adaptive. Data outside the critical difference threshold detects (i) injury and/or illness; or (ii) an increased risk for injury and illness. Ongoing research utilizing the systems and methods disclosed herein indicates a model of reliable prediction of actual injury and illness as well.

Particular embodiments utilize the systems and methods to detect an increased risk for injury and/or illness in a subject. Subjects can include athletes (e.g., training athletes and/or elite athletes); and/or patients (e.g., physical therapy patients; cancer patients; recovering patients) and/or interested individuals, groups and teams who may for whatever reason be interested in detecting risk of injury and/or illness and or tracking, monitoring and maintaining their individual health and wellness, for example in corporate wellness programs, and groups of individuals for example in military training and deployment where each individual may have a different baseline level of fitness and health at entry. In addition, the systems and methods may be applied to individuals in specific environments, especially extreme environments and activities such as expeditions, space, diving, climbing, to keep individuals well and detect risk of illness or injury.

In addition, the systems and methods may be applied to any animals, for example horses, to detect risk of illness, injury and to sustain health and performance. The systems and methods can be used to maximize available training days for subjects by reducing training days lost to injury or illness.

In particular embodiments, the systems and methods can be used to optimize the health of a team, such as a team of elite (e.g., professional or sponsored) athletes. The systems and methods can be used to tailor training programs for individuals on the team and can also assist coaches and managers to better choose players ready to compete on a given day.

### BRIEF DESCRIPTION OF THE SEVERAL VIEWS OF THE DRAWINGS

FIG. 1 depicts the theory of hormesis (prior art).

FIG. 2 depicts a questionnaire that can be used to screen individuals for unexplained underperformance syndrome (UUPS).

FIGS. 3A-3E provide exemplary formats to collect contextual data from individuals. In these FIGS. 3A-3E depicted questions can be presented on the same screen or can be presented in previous or future screens.

FIGS. 4A, 4B depict exemplary oxidative stress (OS) measures over time as compared to an individual's critical difference threshold. FIG. 4C depicts data from an individual with anti-oxidant values outside of the individual's critical difference threshold. An upper respiratory tract infection was observed during this time. Low FORD values have been observed in numerous individuals around the time of an infection.

FIGS. 5A-5I provides exemplary intervention recommendations based on OS test results and associated rules bases.

FIG. 6 provides an example of how the systems and methods can be used to efficiently manage injury and illness risk for a team.

FIG. 7 illustrates an example system for tracking redox homeostasis.

FIG. 8 is a flow diagram of an example process for tracking and managing redox homeostasis.

FIG. 9 is a flow diagram of another example process for tracking and managing redox homeostasis.

FIG. 10 is a flow diagram of another example process for tracking and managing redox homeostasis.

FIG. 11 provides a Case Profile plot of each biomarker over time with mean (black dot), smoothed trajectory (dark grey line) and 95% confidence interval displayed (darkest shaded area).

FIGS. 12A and 12B provide Relative changes (%) from baseline (time point 8:00 AM) for FORT (11A; panel A) and FORD (11B; panel B) over the 10-hours (n=12). Dark black lines denote group average (mean) expressed as percentage change from baseline, with grey lines as individual responses. * Significant effect for FORT (panel A) over time (p<0.001).

FIG. 13. FORT, FORD, and oxidative stress (OS) Index (OSi) at rest, post warm-up, sub-maximal and maximal exercise and into 20 minutes of static recovery (PV adjusted data presented only). Letters (a, b, c) that differ denote significant differences between time points for each respective biomarker (p<0.05). FORT^{a, b} d=0.23-0.32; FORD^{c, b, a} d=0.87-1.55; OS Index^{a,b} d=0.46.

### DETAILED DESCRIPTION

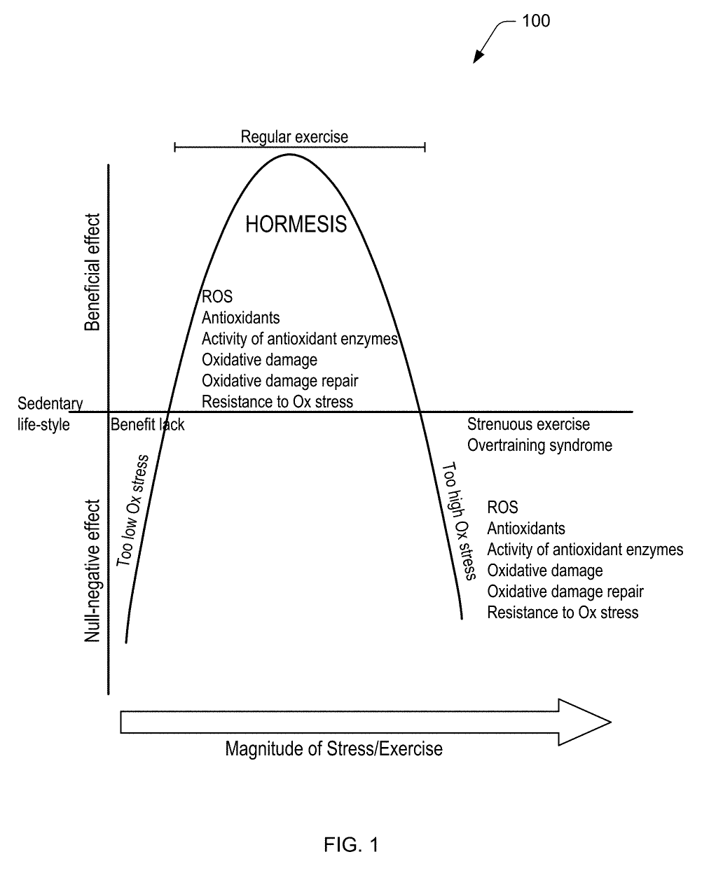

One of the biggest challenges in maintenance and improvement of health is how hard to train, exercise or exert oneself physically while keeping within an acceptable risk of injury or illness. A balance (redox homeostasis) between pro- and anti-oxidants is essential for health, muscle function and training adaptation. This is because alterations away from redox homeostasis (ARH) are associated with degradations in physical performance, illness, and fatigue. The theory of hormesis 100 (see FIG. 1, prior art) best captures this challenge. If the stress on a subject is too high, excessive oxidative stress and maladaptation ensue, whereas if the stress on the subject is too low, there is not enough pro-oxidant activity to result in adaptation, or the rate of adaptation may be blunted. Exercise is known to be a source of reactive nitrogen and oxygen species (RNOS), leading to post-exercise alterations in redox homeostasis. Initially, RNOS were thought to be detrimental to recovery, but it is now clear that RNOS are important for adaptation to endurance training, skeletal muscle hypertrophy and protein signaling.

The current disclosure provides systems and methods to detect injury and/or illness and/or to detect an increased risk for injury and illness by testing regularly for ARH and wellness/health. The systems and methods utilize point-of-care testing to generate serial measures from individuals. In particular embodiments, data from the serial measures can be used to create individualized baselines and thresholds so that an individualized risk indicator is generated. In addition, in particular embodiments, the systems and methods can utilize a rules base to generate alerts, messages, notifications and/or recommendations based on the individualized baseline and trend indicators. In particular embodiments, the alerts, messages, notifications and/or recommendations can be automatic and/or self-generated, and/or manually-created alerts, messages, notifications and/or recommendations.

The systems and methods detect risk of injury and/or illness. When an increased risk for injury and illness are detected, interventions can be put into place to reduce this risk. Similarly, where good health is indicated, a rules base may generate alerts, notifications and recommendations to maintain and sustain good health status and maintain status within the particular subject's individualised range and/or recommended range. Moreover, the systems and methods can incorporate contextual data from individuals that allow better detective modelling and more contextual alerts, messages, notifications & recommendations.

The current disclosure describes new information on the analytical, biological variation (BV), index of individuality (II), critical difference value (CDV) for markers of oxidative stress (OS) and nutritional status, training load and sleep. In particular embodiments, this new information can be used for monitoring and assessing meaningful changes in serial results in individuals in relation to health, exercise, and performance. The importance of generating such data is stressed, given that for many biomarkers (notably redox measures), laboratory reference ranges can have poor utility due to BV and other factors.

The disclosed systems and methods provide a unique framework for managing training load and recovery requirements in subjects. In particular embodiments, the systems and methods can be used to detect risk of maladaptation, fatigue, illness, and/or injury. In particular embodiments, where additional contextual information is gathered on the individual, the systems and methods can be used to identify individual tolerances to training loads and to identify different risk factors for injury and illness in different individuals. For example, the systems and methods can track that one individual is more prone to ARH following air travel than another individual. The systems and methods can also identify that one individual is more prone to ARH following poor dietary intake than another individual. In this manner, the systems and methods allow adaptive implementations to reduce the risk of ARH. In particular embodiments, the systems and methods can be used to minimize training days lost to injury and/or illness.

In particular embodiments, the systems and methods can be applied in sports and clinical practice and in the field (e.g. training camps) for the assessment of ARH.

In particular embodiments, the systems and methods can be applied with rehabilitation following a surgical intervention or other therapy or treatment.

In particular embodiments, the systems and methods can be applied with cancer patients and cancer survivors who are encouraged to use exercise as means of recovery of function. This is particularly the case as oxidative stress is a feature of cancer. Early on in treatment and recovery, there is a spike in OS and poor exercise tolerance with high fatigue. The systems and methods disclosed herein can be used to guide exercise therapy in the rehabilitation of cancer patients (aiding recovery and compliance and nutritional strategies), and grade the exercise treatment better to the individual.

In particular embodiments, in practice, the systems and methods: (1) collect contextual data from an individual; (2) collect a sample from the subject; (3) analyze the sample for ARH; (4) utilize the ARH sample results to calculate an initial baseline; (5) collect additional samples from the subject; (6) analyze the additional samples for ARH; (7) utilize the ARH samples results to calculate a steady state baseline; (8) collect and analyse additional samples from the subject; (9) generate critical difference threshold values for the individual; (10) collect and analyse additional samples from the subject; (11) compare results from the most recent analysis to critical difference threshold values generated for the individual; and (12) determine whether the individual is at heightened risk for illness and injury based on the comparison.

In particular embodiments, in practice, the systems and methods: (1) collect multiple samples from a subject serially or in real-time in time under steady state conditions; (2) analyze the samples for ARH; and (3) utilize the ARH sample results to calculate critical difference values for the individual.

In particular embodiments, the systems and methods (1) collect multiple samples from subjects serially under steady state conditions, fasted, standardizing each collection for the time of day; (2) analyze the sample for ARH; (3) collect the aforementioned contextual data from the subject to ascertain health status; and (4) utilize the ARH sample results to calculate critical difference values for the individual subjects.

In particular embodiments, the systems and methods can be used by any individual to monitor, track and help sustain maintenance of a desired level of wellness and health. For example, the systems and methods can be utilized as part of a corporate wellness program. An individual who is particularly interested in their personal health may also use the systems and methods described herein.

In particular embodiments, in practice, the systems and methods: (1) collect contextual data from an individual via reported, self-reported, automatically generated data, third-party data, wearables, mobile devices, and environmental conditions; (2) collect a sample from the subject via an external, implanted, ingested, patched, wearable or other device; (3) analyze the sample for ARH whether within the sample collection method, remotely or wirelessly transferred, or in any external analyzer; (4) utilize the ARH sample results to calculate an initial baseline; (5) collect additional samples or continuously report sample data from the subject; (6) analyze all samples for ARH; (7) utilize the ARH samples results to calculate a steady state baseline; (8) collect additional samples or continuously report sample data from the subject; (9) generate critical difference threshold values for the individual; (10) collect and analyse further samples from the subject; (11) compare results from the most recent analysis to critical difference threshold values generated for the individual; (12) determine whether the individual is at heightened risk for illness and injury based on the comparison; and (13) determine the level of wellness of the individual based on the comparison.

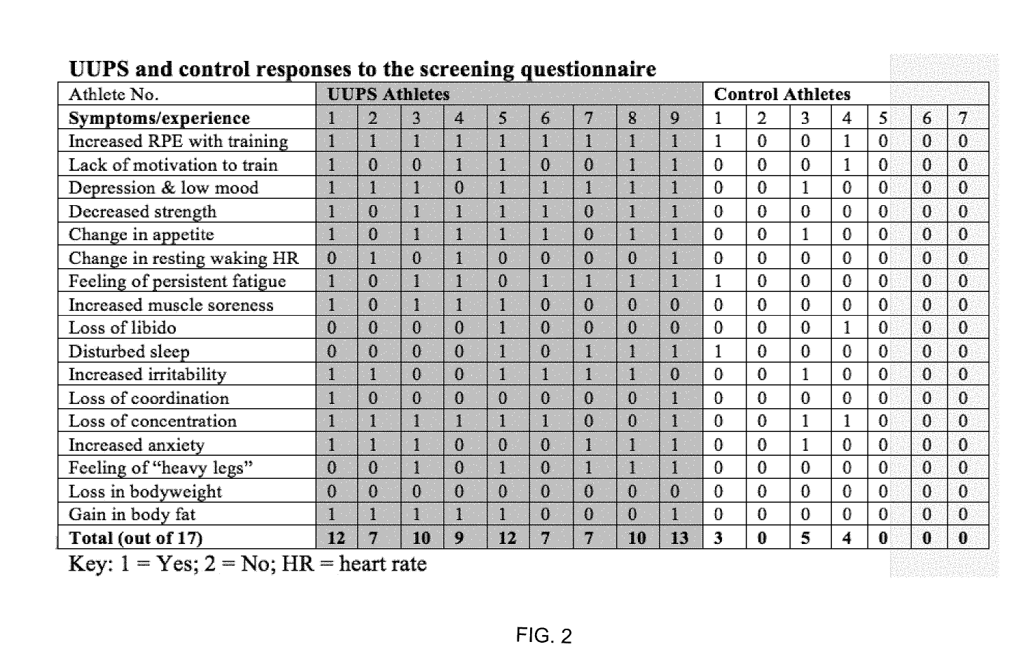

Particular embodiments detect an increased risk of illness. As used herein, illness refers to non-functional overreaching (NFOR), overtraining syndrome (OTS), and/or unexplained underperformance syndrome (UUPS). NFOR refers to an accumulation of training and/or non-training stress resulting in a short-term decrement in performance capacity with or without related physiological and psychological signs and symptoms of maladaptation in which restoration of performance capacity takes several days to weeks. Cardoos, Overtraining Syndrome, Current Sports Medicine Reports, 157 (2015). OTS is the same as NFOR, except that the decrement is more long-term, with restoration of performance levels taking several weeks or months. Cardoos, supra. UUPS is similar to OTS, but reflects the complexity of these maladaptive syndromes and their more multifactorial etiology. Lewis et al., BMJ Open Sport Exerc Med 2015; 1:e000063. Doi:10.1136/bmjsem-2015-000063. For example, UUPS acknowledges that an imbalance between training load and recovery may not always be the primary cause for underperformance. Lewis et al. supra. Underperformance can similarly be due to, e.g., a significant life stressor, poor nutrition, excessive travel, or other contextual factors described herein. FIG. 2 depicts a questionnaire that can be used to screen individuals for unexplained underperformance syndrome (UUPS). A score of 7 or higher is indicative of UUPS. Predicting the risk for illness, as used herein, does not include predicting risk for a pathogen-based sickness due to encounter with an environmental pathogen. The systems and methods disclosed herein can, however, be used to track physiological recovery from a pathogen-based sickness.

Aspects of the disclosure are now described in more detail.

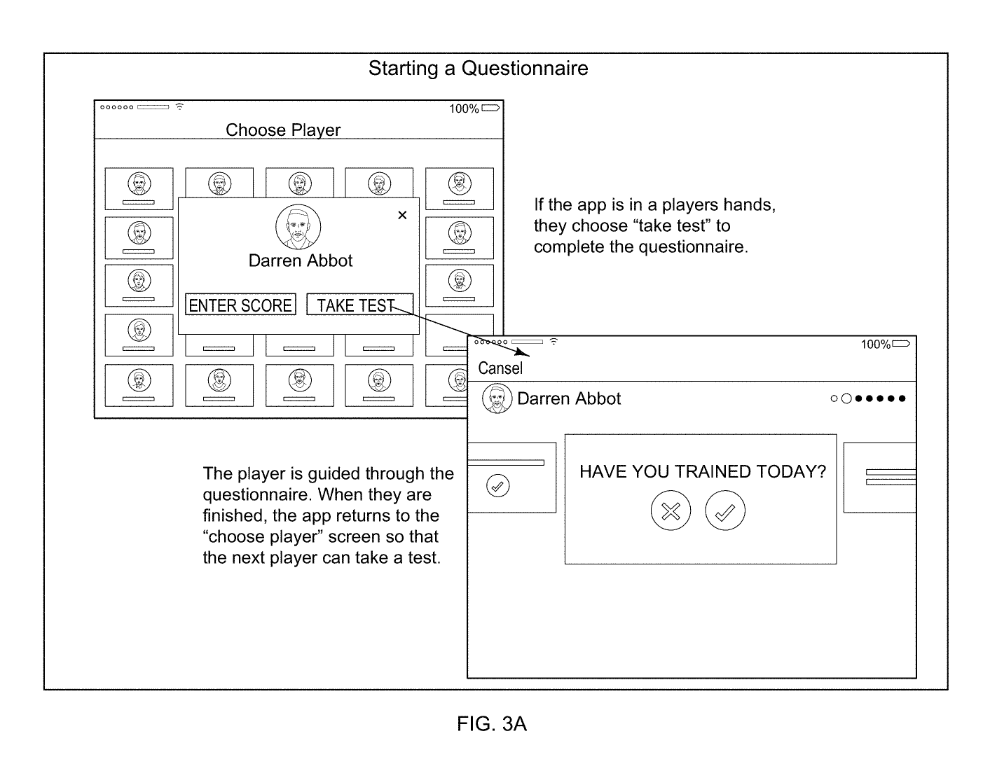

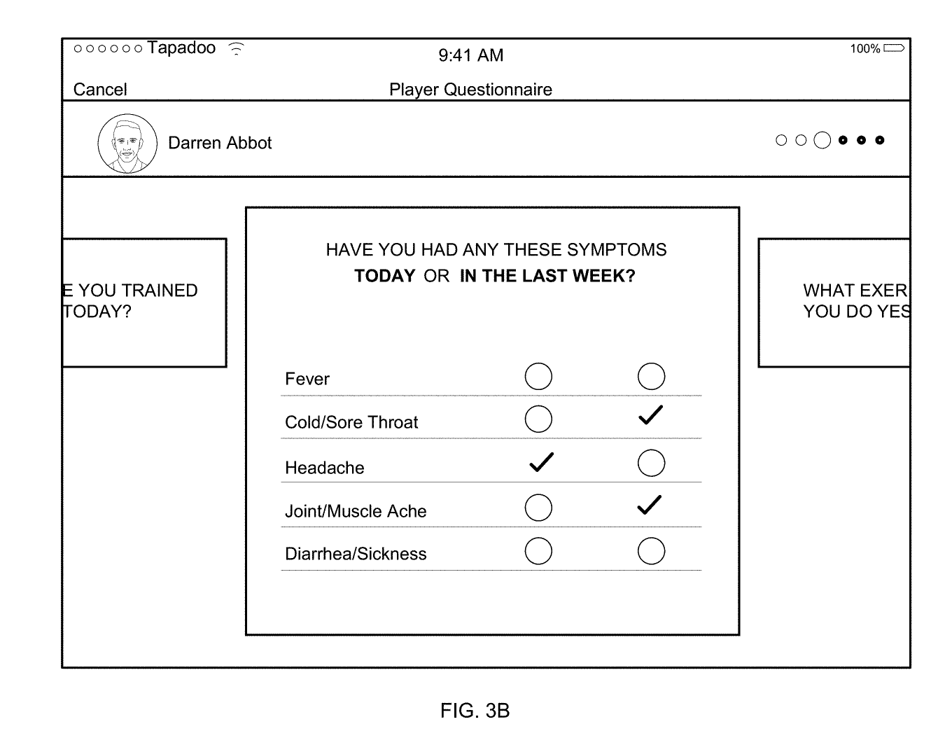

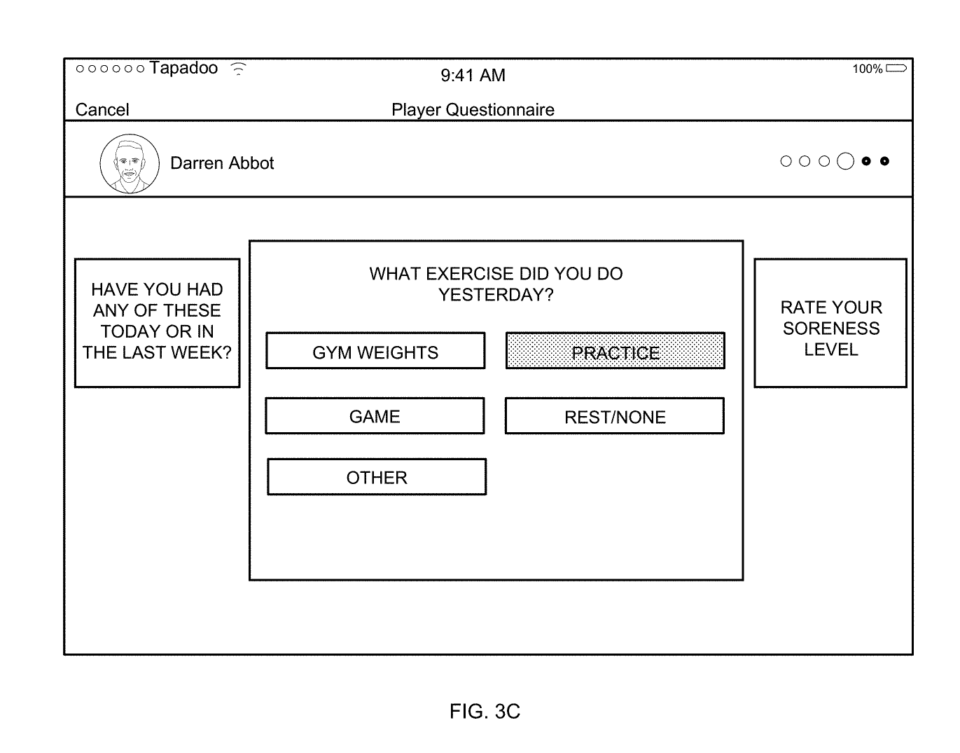

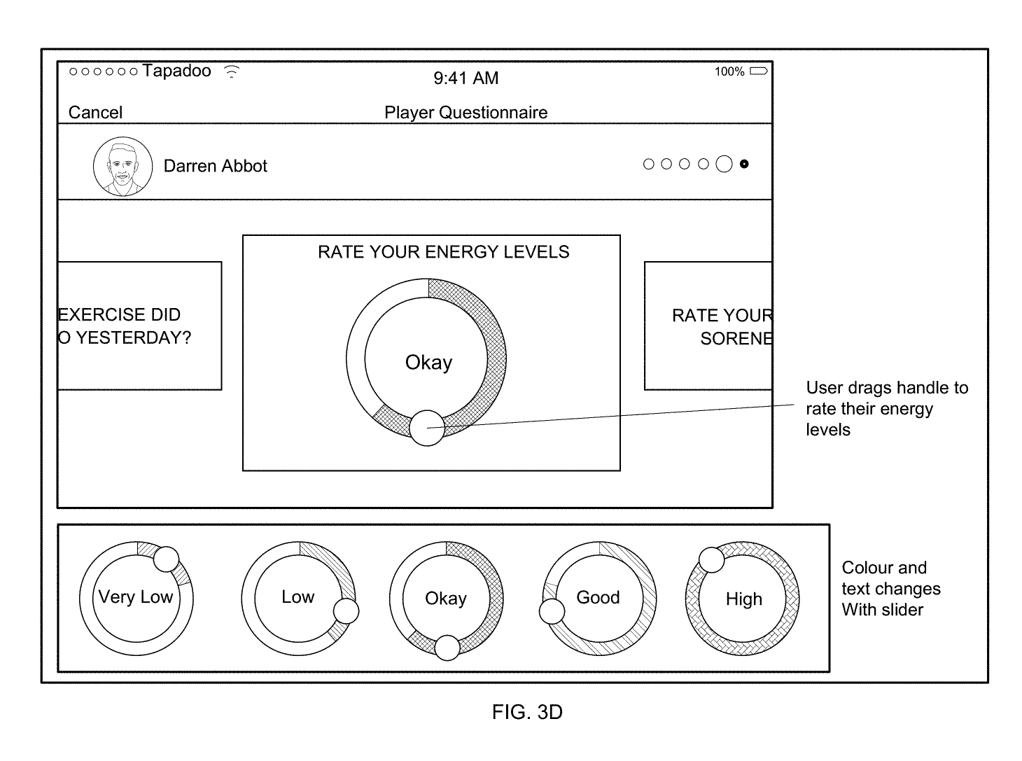

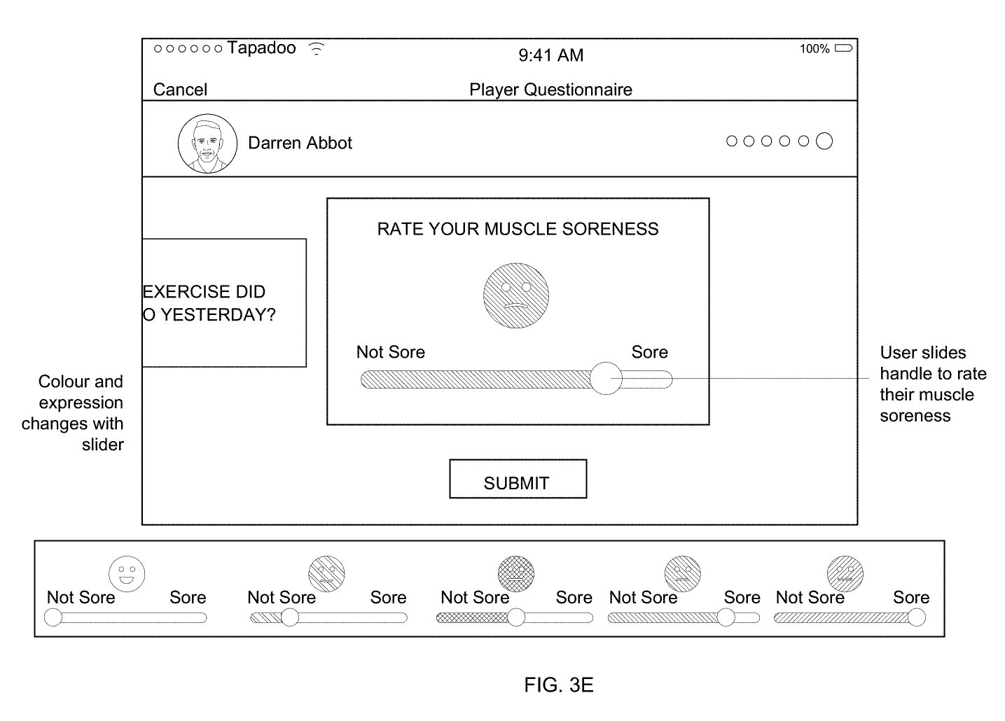

Contextual Data. Particular embodiments collect contextual data from individuals to allow better detection of injury or illness risk over time and to identify and track internal and external triggers, conditions, symptoms and factors that may affect ARH. Indeed, the collection of contextual data allows for the more accurate interpretation of whether the subject is healthy (e.g. free from an acute illness), and thus whether the redox data collected should be included in the continued calculation of the subject's critical difference threshold over time. In other words, such contextual data can allow the learning of stress and defense triggers in individuals. This feature allows more individualized care and intervention when a subject shows ARH. Types of contextual data to be collected can include: dietary habits, rest habits, current mood, energy levels, goals, injury status, muscle soreness, presence of symptoms suggestive of acute illness etc. The data can be collected by, for example, verbal, written, or computerized questionnaires. FIGS. 3A-3E provide exemplary contextual data questionnaires. As can be seen, contextual questions can be presented in “Yes/No” formats; multiple choice formats; open-ended formats; sliding scales; interval scales, etc.

Physiological Data. Physiological samples are collected from subjects. Any appropriate physiological sample from a subject can be collected and analyzed. Representative sample types include blood, saliva, urine, tear, DNA, perspiration, extracellular fluid etc. Physiological samples can be processed according to procedures well known to those of ordinary skill in the art.

In particular embodiments, physiological samples include blood samples. In particular embodiments, physiological samples can be low friction blood samples. In particular embodiments, blood samples can be whole blood capillary samples. In particular embodiments, blood samples can be taken from the ear lobe or fingertip. In particular embodiments, blood samples can be taken from the antecubital vein.

Pro-oxidant and Anti-oxidant values can be determined from the physiological samples. For example, in particular embodiments, samples are analyzed for ARH utilizing a free oxygen radical test (FORT) and a free oxygen radical defense test (FORD).

The free oxygen radical test (FORT) and free oxygen radical defense test (FORD) assays provide an accurate and non-invasive method for monitoring ARH, with excellent reliability and repeatability. FORT captures the concentration of hydroperoxides in a physiological sample, being derived from numerous lipid and protein molecules, ubiquitous within human tissues. For example, FORT detects lipid hydroperoxides derived from phospholipid, cholesterol and fatty acids, and protein hydroperoxides from proteins, peptide, amino acids, DNA and nucleic acids. Hydroperoxides are fairly stable, with protein and peptide hydroperoxides said to have a half-life of several hours at room temperature. Furthermore, the peroxidation of proteins and formation of protein hydroperoxides is the most extensive modification by radicals, and exceeds the formation of more commonly used biomarkers of protein oxidation such as protein carbonyls under similar conditions.

In particular embodiments, FORT is a colorimetric assay based on the capacity of transition metal ions (Fe³⁺/Fe²⁺) to catalyze the breakdown of hydroperoxides (R—OOH) into derivative radicals [alkoxyl (R—O^{.}) and peroxyl radicals (R—OO^{.})] within the physiological sample. For example, the application of an acidic buffer to a 20 μL capillary sample, releases the transition metals from associated proteins, which react with the hydroperoxides present in the sample, producing the alkoxyl and peroxyl radicals. The derivative radicals are trapped through the addition of a buffered chromogen (reagent; an amine derivative, CrNH₂) and develop into a radical cation in a linear based reaction at a controlled temperature of 37° C., photometrically detectable at 505 nm.

R—OOH+Fe²⁺→R—O^{.}+OH⁻+Fe³⁺

R—OOH+Fe³⁺→R—OO^{.}+H⁺+Fe²⁺

RO^{.}+ROO^{.}+2CrNH₂→RO⁻+ROO⁻+[Cr—NH₂^{+.}]

The intensity of the sample color correlates with the quantity of radical compounds and therefore the concentration of hydroperoxides in the physiological sample, according to Lambert-Beer's law. The results are expressed as equivalent concentrations of H₂O₂ mmol·L⁻¹ and linearity ranged from 1.22 to 4.56 mmol·L⁻¹ H₂O₂.

FORD assay. The FORD test is an estimation of plasma anti-oxidant capacity, with the water-soluble molecules of ascorbic acid, glutathione, and albumin (but not uric acid), accounting for the majority of anti-oxidant activity (Palmieri & Sblendorio, 2007. Eur Rev Med Pharmacol Sci 11: 309-342).

The FORD test determines the presence of plasma anti-oxidants via a colorimetric assay based on the capacity of the sample to reduce a preformed radical cation. In the presence of an acidic buffer and a suitable oxidant (FeCl₃), the chromogen that contains 4-amino-N,N-diethylaniline sulfate forms a stable and colored radical cation, photometrically detectable at 505 nm. The anti-oxidant compounds present in the plasma sample reduce the radical cation of the chromogen, quenching the color, and causing a discoloration of the sample, proportional to the concentration of anti-oxidants present. The absorbance values generated are compared to standard curves derived from Trolox (6-hydroxy-2,5,7,8-tetramethylchroman-2-carboxylic acid), a derivative of vitamin E with enhanced water solubility. FORD values are reported as Trolox equivalents, mmol·L⁻¹, linearity ranged from 0.25 to 3.0 mmol·L⁻¹ Trolox.

Chromogen(uncolored)+oxidant (Fe³⁺)H⁺→Chromogen.+(color)

Chromogen.+(color)+AOH→Chromogen+(uncolored)+AO

In particular embodiments utilizing FORD and FORT, the ratio of the FORT and FORD tests provides an index of OS (an Oxidative Stress Index (OSi). The FORT-FORD Test (FFT) is a POC test that can be undertaken rapidly with subjects, for example, athletes in a training environment.

Critical Difference Thresholds and Ranges. Results from the physiological testing are used to generate critical difference values for individuals in the form of critical difference thresholds and ranges. A threshold is a score an individual should not go above or below as appropriate. For example, a pro-oxidant score should not exceed a threshold value and an anti-oxidant score should not fall below a threshold value. A range is a numerical range or band between two thresholds.

Initially, there is not enough historic data available for an individual to calculate the individual's critical difference values. In this case, in particular embodiments, initial critical difference values can be based on clinical values and ranges published by a manufacturer of a redox testing system used with a subject of similar demographics (i.e. healthy and gender matched). In particular embodiments, data representing a larger population may be imputed to the subject and used to determine initial critical difference values. In one example, a Bayesian model, or any model or approach taken for generating reference ranges where the approach enables the reference ranges to adapt and be updated from data collected longitudinally and serially or in real-time may be applied using the data from a larger population as a prior input in determining critical difference values for an individual subject. That is, data associated with a larger population of subjects is used to provide initial estimates of the standard deviation of the biomarker. This estimate may be updated sequentially as more data for the subject in question are gathered.

The distributions assumed for each parameter in the model are shown below as is the procedure to generate the reference range. According to one example of the Bayesian model, there is no closed form solution for the double integral, in which case, it may be solved using a Markov Chain Monte Carlo sampler, for example.

Generating an initial reference range for an individual may take the form of Equation 1, shown below. In particular embodiments, for assumptions, let
i=1, . . . , M indicating the individuals
j=1, . . . , nᵢ indicates the measurement occasion for individual i
Then Xᵢⱼ is the biomarker value for person l at time j. In this example it is assumed that:
Xᵢⱼ|μᵢ,σᵢ²˜N(μᵢ,1/σ²)
μᵢ|m, τ²˜N(m, 1/τ²)
m˜N(0, 1000)
τ²˜Gamma(0.001,0.001)
σ²˜Gamma (0.001, 0.001),
The reference range for individual/may be determined by Eq. 1, below.

P(Xᵢ,new|data)=∫∫P(Xᵢ,new|μᵢ,σᵢ²)P(μᵢ,σᵢ²|data) Eq.1

Thus, for each biomarker, the number of subjects (M) and the number of points for each subject (n) are used in order to determine the distribution for that biomarker for a particular subject. This model assumes that the biomarker follows a normal distribution with some degree of variability.

According to some embodiments, a Bayesian model or other statistical adaptive model, such as the one described above, can be used to initially determine the reference range and can also be used as an initial critical difference threshold, and as additional values are available through subsequent sampling of a subject, create critical difference values for a particular subject by incorporating the new information and updating the parameter settings of the model.

Returning to the individual at the beginning of sampling, when a subject is repeatedly in a steady state (e.g., healthy and in a fasted, hydrated and rested state) at the time of testing, a steady state base may be established for the individual, such as by conducting at least three consecutive tests taken under a defined steady state. A steady state baseline for a subject is helpful in order to calculate the individual critical difference threshold for the subject.

In particular embodiments, the steady state baseline is calculated as the average of three measurements taken under steady state. In particular embodiments, the steady state baseline can be calculated as the average of at least two measurements taken under steady state. In particular embodiments, this baseline can then be updated as a rolling average over the last 7 measurements in order to capture changes from baseline still under steady state. In particular embodiments, this baseline can then be updated as a rolling average over the last, for example, 3-15 measurements in order to capture changes from baseline still under steady state. If any data is recorded while the subject is sick or injured, this data can be excluded from the steady state baseline calculation, to avoid the introduction of confounding factors.

In particular embodiments, steady state critical difference values (e.g., individual thresholds) are established for each subject. In particular embodiments, the steady state critical difference thresholds can be calculated as marker specific multiples (e.g. 1.17) of the steady state baseline. The critical difference threshold for a subject may be based on a Bayesian adaptive model that incorporates the subject's correlation over time, and may additionally incorporate a larger population in establishing threshold values. This is a more advanced version of calculating the steady state critical difference thresholds.

New data can be included in a database containing a subject's past data. These data can then be used to generate thresholds and reference ranges, which may be based on a fixed multiple of a smoothed estimate (using a running mean and/or a lowess smoother) weighted towards more recent (i.e. acute phase) observations. A second set of thresholds and reference ranges can be generated that incorporates the within-subject variability overtime to generate an individualized multiplier.

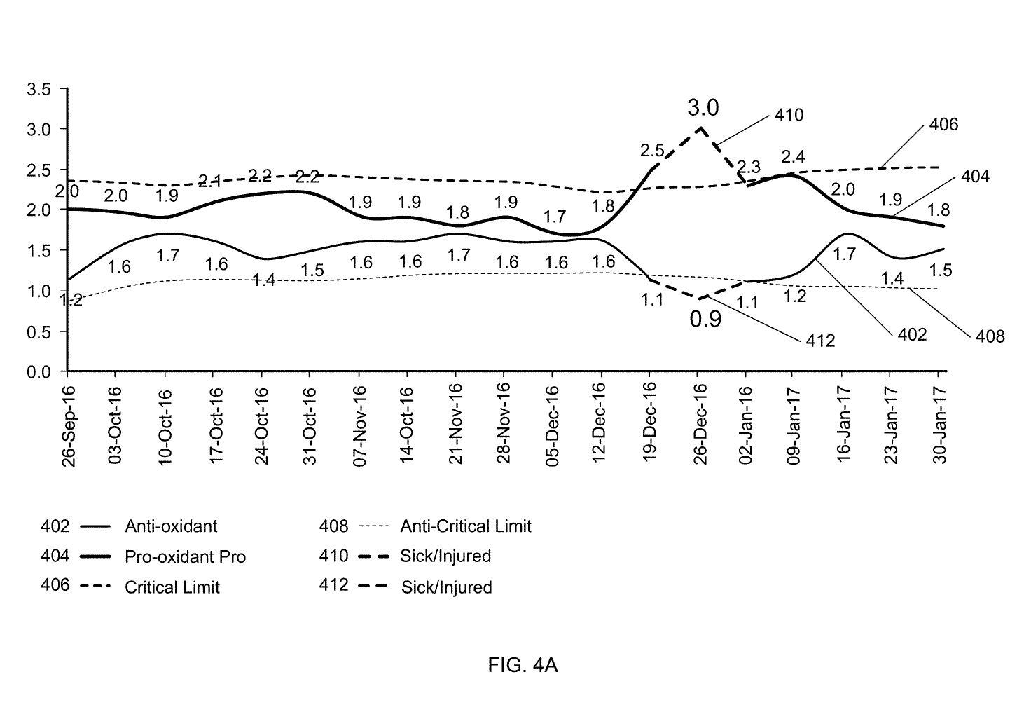

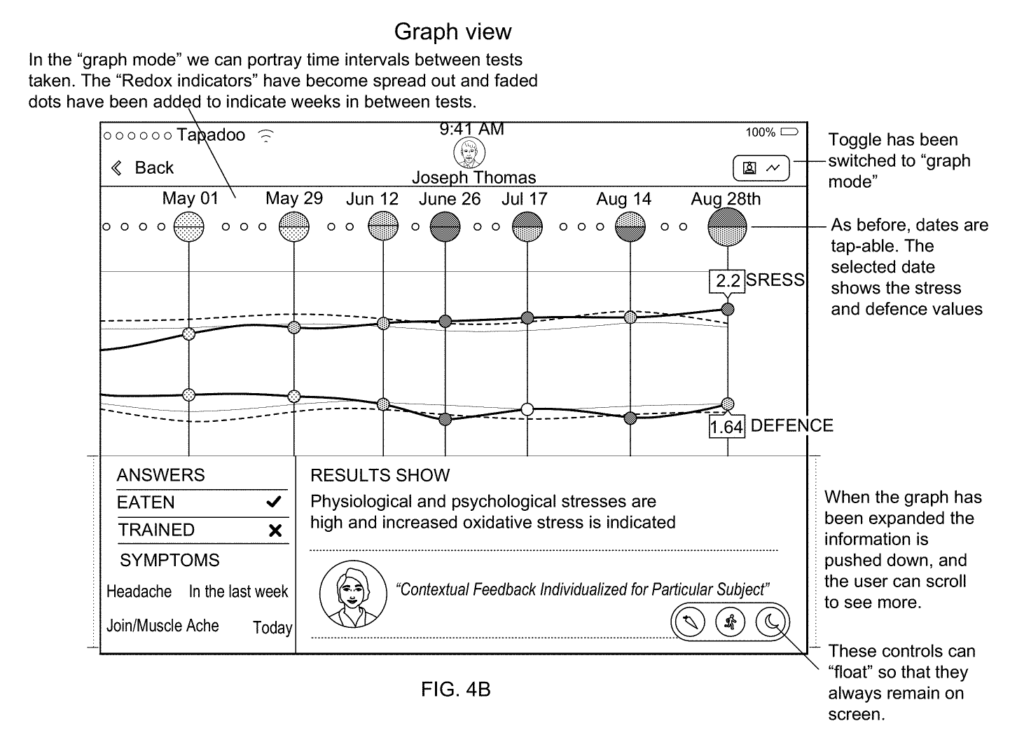

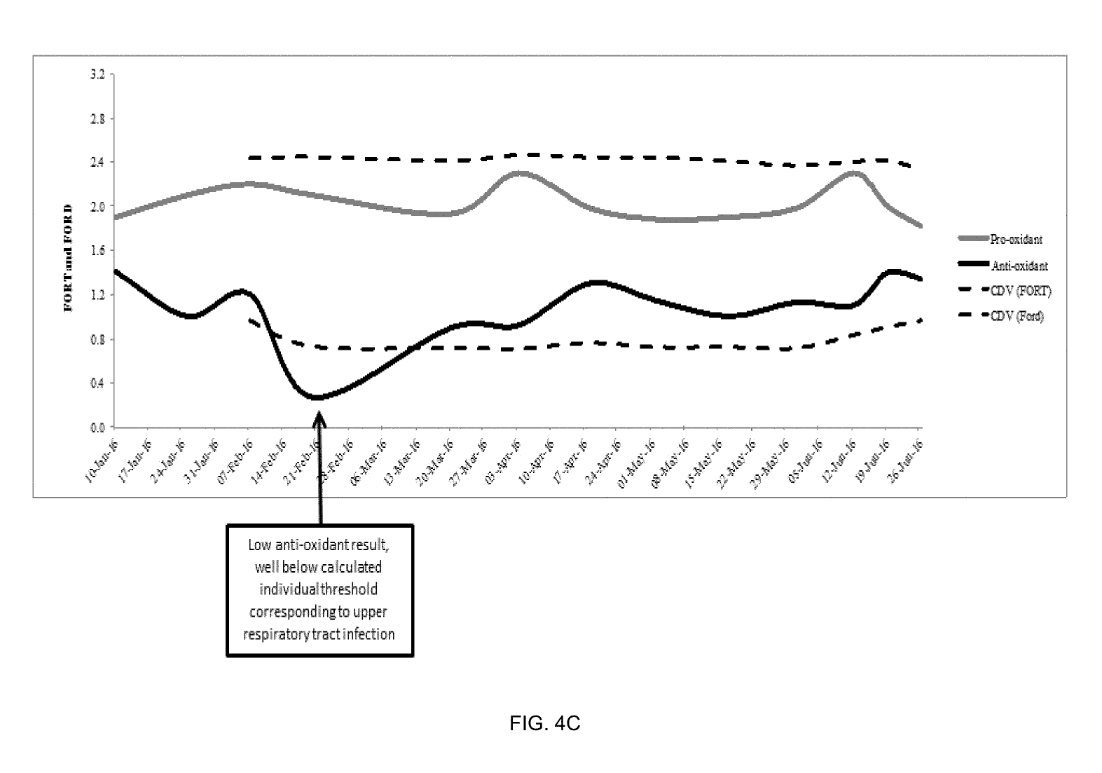

FIGS. 4A and 4B depict what an output of these processes could generate for a particular individual. FIG. 4A illustrates a sample graph showing anti-oxidant values 402 and pro-oxidant values 404 for a subject over time. These values are measured as described herein, such as by conducting FORD and FORT assays, in this example, once a week for a period of 4 months. Based upon the historical values, represented by lines 402 and 404, a critical difference threshold has been calculated for this subject. The critical difference threshold is indicated on the high side, by Pro Critical Limit 406, and on the low side by Anti Critical Limit 408. Interestingly, where the measured values fall outside the critical difference threshold, such as at 410 and 412, these values coincide with periods of ARH where the subject is at increased risk for injury and illness. Thus, by creating a critical difference threshold and comparing biological marker values to the critical difference threshold, a subject's increased risk for injury and illness can be detected and appropriate interventions can be implemented.

FIG. 4B provides more detail than FIG. 4A and depicts how the information could be beneficially shared with a user. In this depiction, each sample time point is associated with a pie chart that readily shows color-coded results for pro- and anti-oxidant results. In this example, if both values are within range, both halves of the pie chart are light grey (in color, for example, they could be green to indicate “proceed; no problems”). If pro-oxidant levels are out of range, the top half of the pie chart is dark grey (in color, for example, this could be red to indicate “stop; warning”). If anti-oxidant levels are out of range, the bottom half of the pie chart is dark grey. If both pro- and anti-oxidant levels are out of range, the entire pie chart is dark grey. In this depicted example, the light dots between each pie chart denote different passages of time between testing dates. This example also shows how contextual data can be used and incorporated.

Stated another way, the systems and methods disclosed herein can generate a Stress Score and a Defense Score for an individual, which can be used to detect risk of injury and illness in the subject. In particular embodiments, a combination of points (e.g., thresholds) establish the band or status (i.e., the range) the pro-oxidant and anti-oxidant values fall in between (e.g. between X and Y=In Range).

TABLE 1
Correspondence between Critical
Difference Range and Stress Score

| Critical Difference Range | Stress Score (FORT Level) |
|---|---|
| Between X to Y | In range |
| Higher than Y | High |
| Lower than X | Low |

TABLE 2
Correspondence between Critical Difference
Range and Defense Score

| Critical Difference Range | Defense Score (FORD Level) |
|---|---|
| Between X to Y | In range |
| Higher than Y | High |
| Lower than X | Low |

If the pro-oxidant score falls above the individual critical difference threshold, this would be deemed to be a subject who is not coping (in a physiological sense) with the combined ‘load’ of physical and mental stress. The subject is at a higher risk of injury and illness at this point. Similarly, if the anti-oxidant score falls below the individual critical difference threshold, then a subject's recovery from a “stressor” may be compromised and a timely intervention can be suggested and administered to augment recovery.

In particular embodiments, the rules base can include five primary sections/rules, though may be completed in fewer or more sections/rules:
1. Using the critical difference ranges, the rules base establishes and lists the number of statuses a pro-oxidant value (e.g., FORT) and anti-oxidant value (e.g., FORD) can fall under (e.g. outside individual threshold, trending towards individual threshold, rapid spike, and so on).
2. The rules base lists the different combinations of pro-oxidant and anti-oxidant statuses.
3. For each combination of statuses, the rules base indicates whether an alert is required or not.
4. The rules base determines the insight/message associated with each alert (e.g. ‘Significantly raised X is indicated’).
5. The rules base determines the accompanying information/recommendations for each alert (e.g. potential outcomes and action).

In particular embodiments, pro-oxidant values and anti-oxidant values can be calculated as a ratio score. In particular embodiments, FORD and FORT values can be calculated as a ratio score which can be referred to as an Oxidative Stress Index (OSi).

In addition to determining the risk indicators, one or more alerts may be provided to the subject or the clinician based upon the actual values compared with the critical difference threshold. In particular embodiments, the steps to determine how an alert is generated may include:
1. Individual thresholds are determined.
2. The band or status that each pro-oxidant and anti-oxidant value falls under is established.
3. The combined pro-oxidant/anti-oxidant status is established.
4. This combined status determines which alert to generate.

Alerts provide warnings indicating a need to manage training load and recovery requirements of a subject or guidance on maintaining load and recovery. In addition, alerts and messages are targeted depending on the proximity of the values to the individual thresholds and therefore the severity of the indicator. In particular embodiments, when anti-oxidant levels are low, Nutrition and Sleep are listed for review. In particular embodiments, when pro-oxidant levels are high, Nutrition, Sleep, and Recovery are listed for review.

Advice related to nutrition can include a directive to eat nutritionally balanced and periodized meals and snacks, which may include for example, appropriate amounts of proteins, carbohydrates, fats, fruits, vegetables, nuts, and seeds.

Advice related to sleep can include for example, for the individual to aim for a specific number of hours in bed with specific environmental sleep hygiene (e.g., lights out every single night), together with advice on remedial action to take if sleep is disrupted, e.g. find time to nap after times of exertion (e.g., practice, therapy).

Advice related to recovery can include supplementation strategies that can aid recovery, and should be individualized and periodized around periods of stress according to individualized data. For example, advice related to recovery can include: removing any unnecessary additional activity that might compromise recovery; spending time in natural daylight (i.e., outside) to promote a natural cycle of hormones (particularly melatonin), helping to regulate ability to sleep and anti-oxidant defenses and aiming to find an hour every day, and preferably in the morning, to be outside in natural light.

While not depicted in FIG. 4B, it can also be useful to include a depiction related to contextual information to visualize potential events or stressors associated with trends or changes in pro- and anti-oxidant levels. For example, information related to sleep, travel, nutrition, the occurrence of life events, etc. could be indicated on the screen in a manner that associates the timing of one or more of these events with the tracked pro- and anti-oxidant levels.

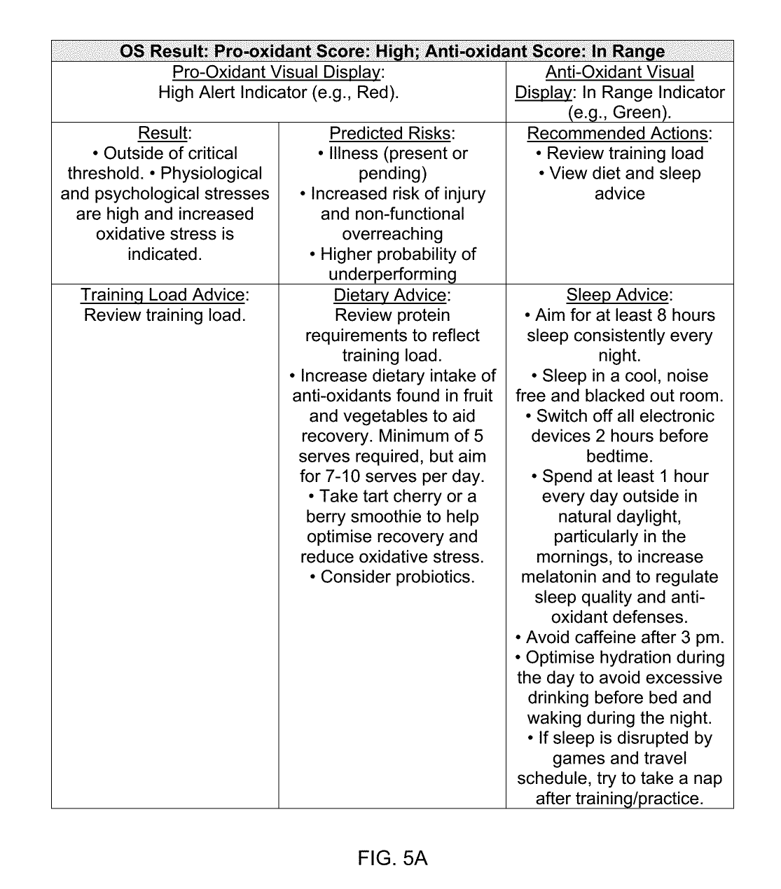

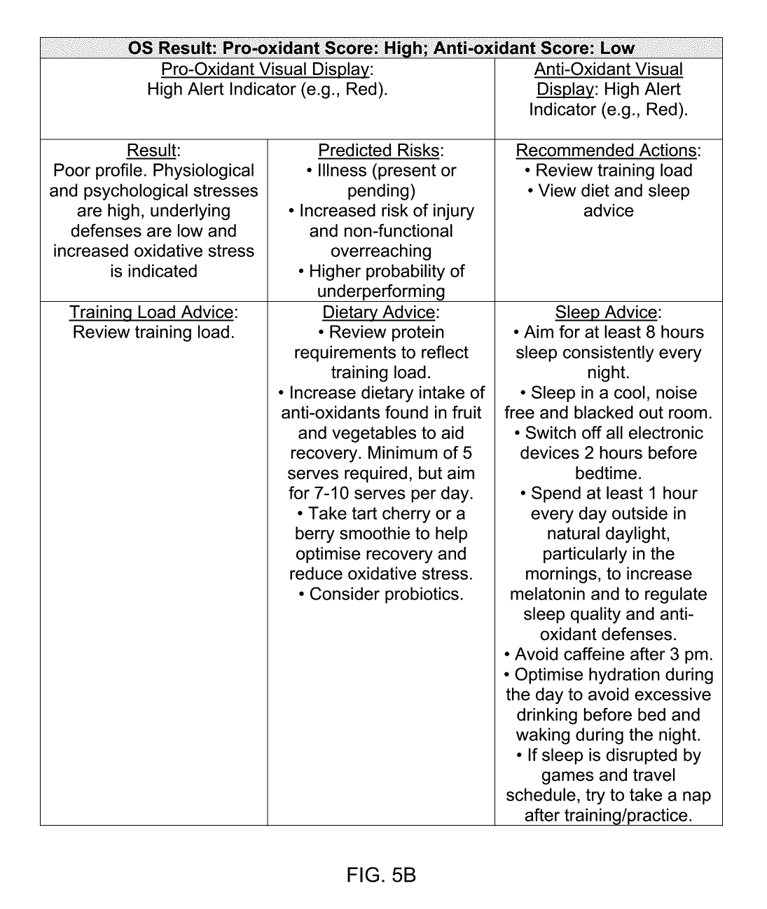

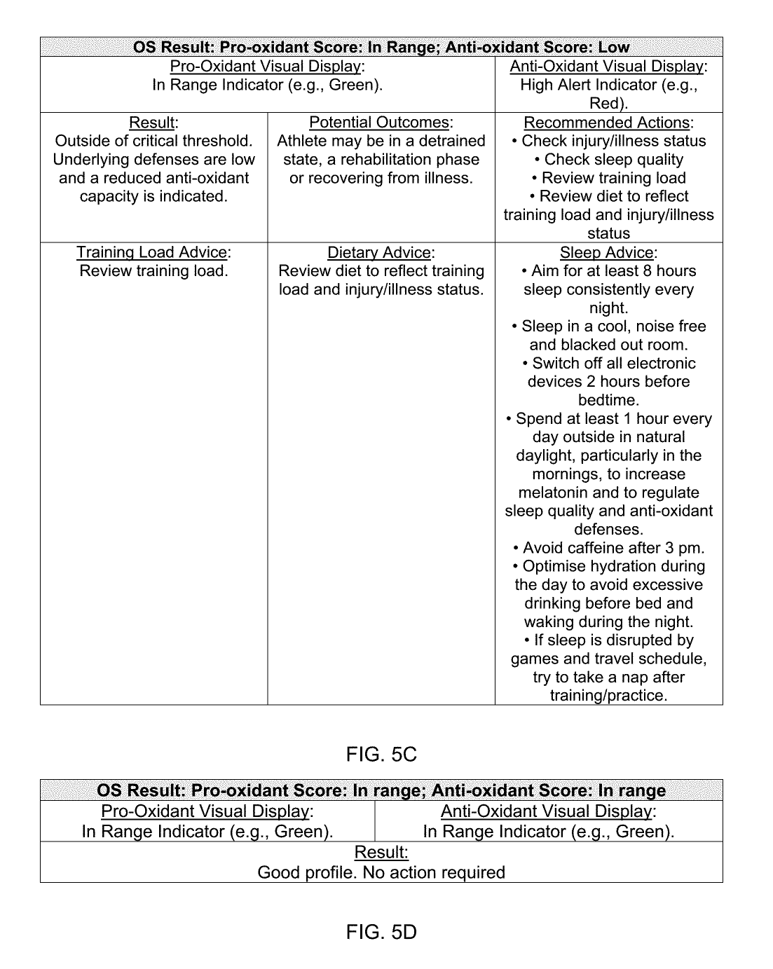

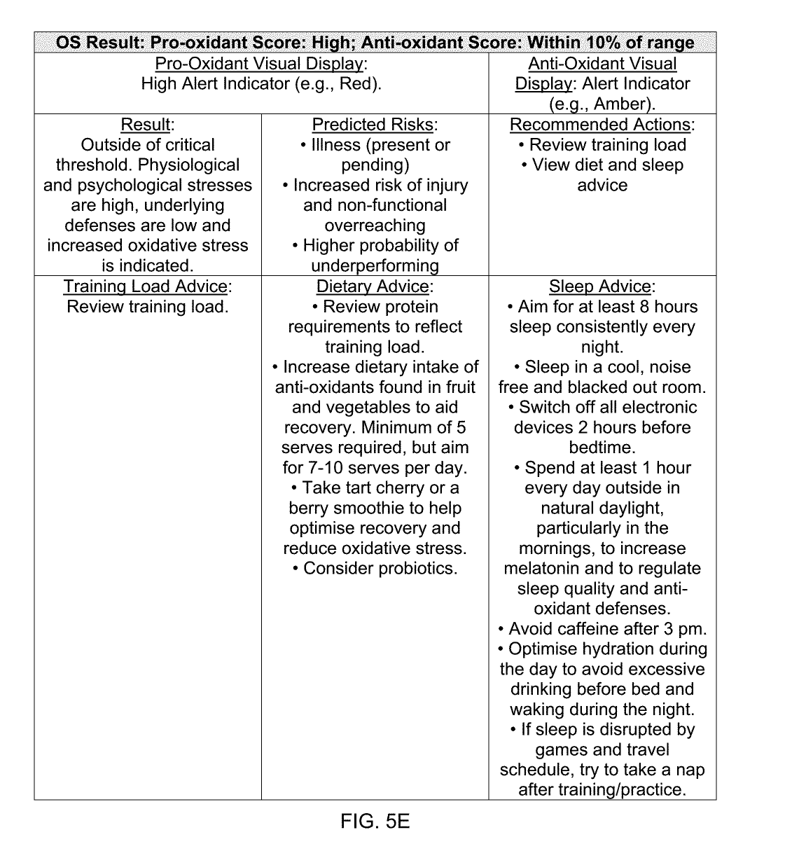

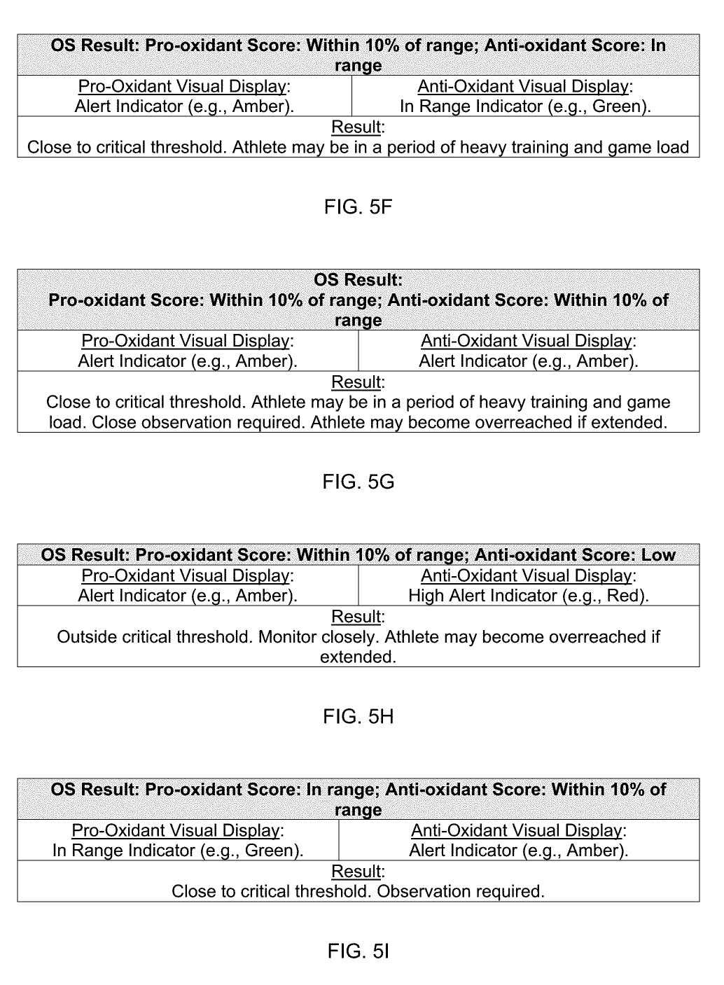

FIGS. 5A-5I provides additional exemplary detail regarding exemplary recommendation rules.

The described systems and methods have shown that (1) the pro-oxidant value can increase and the anti-oxidant value can decrease with increasing cumulative training volume, and (2) when the critical difference threshold is exceeded, injuries and illnesses can occur more frequently, effectively providing a dashboard warning light, e.g. that training load must decrease while physiological adaptation (i.e. rest, regeneration and recovery) occurs and the pro-oxidant and anti-oxidant values normalize.

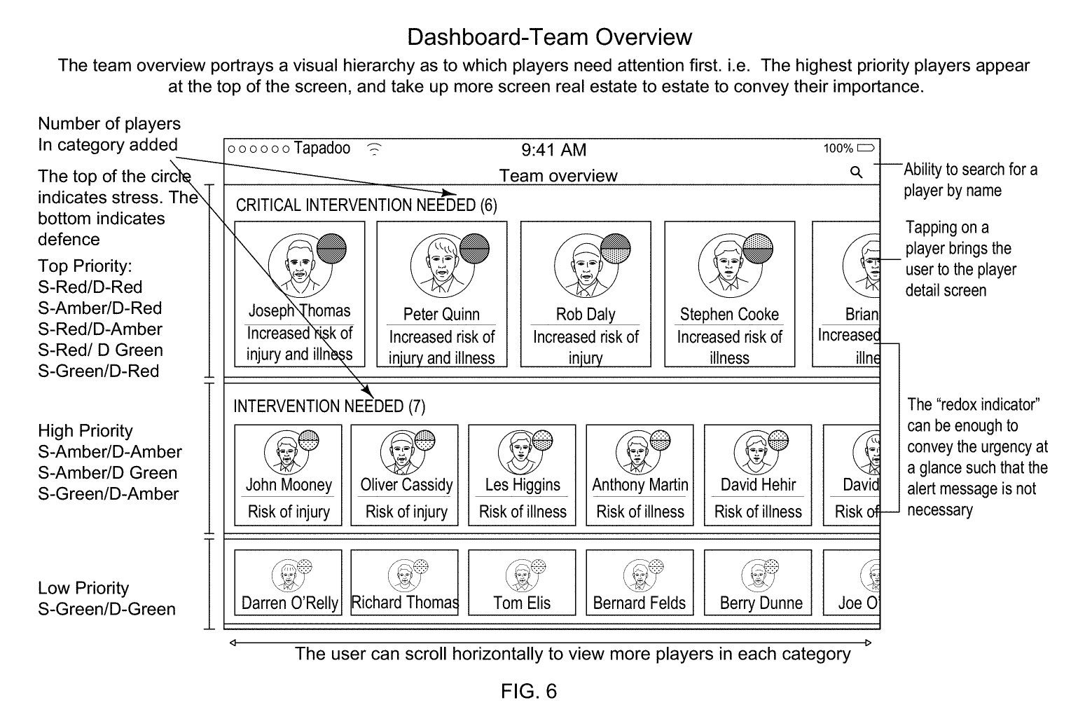

FIG. 6 provides an example of how the systems and methods described herein could be used to efficiently manage injury and illness risk for a team. In this depicted embodiment, team members with the highest risk of injury and illness are brought to the top of the screen for immediate attention and intervention (top priority). Team members with a more moderate risk of injury and illness are also highlighted for attention (high priority). Team members with scores within their critical difference thresholds are not highlighted and can undergo normal training (intervention based on ARH not required). Understanding which players are at highest risk for injury and illness allows coaches to reduce this risk with interventions and can also help with game day and competition rosters.

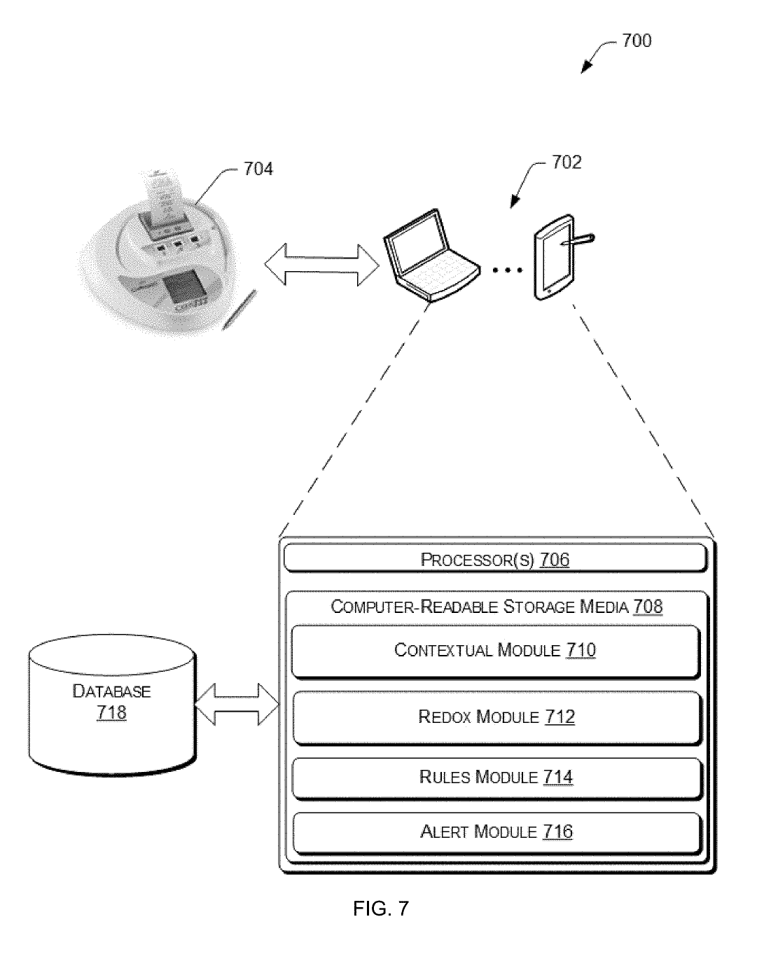

FIG. 7 illustrates a sample system 700 for tracking and managing redox homeostasis as disclosed herein. In particular, a computing device 702 may be used to analyze physiological sample results and display subject data, which may include alerts, or recommendations. The computing device 702 may be implemented as any number of computing devices, including a personal computer, a laptop computer, a portable digital assistant (PDA), a mobile phone, a tablet computer, and so forth. Additionally, the computing device 702 may be combined with a sample analyzer device 704 to provide a specific purpose computing device that both analyzes a physiological sample and provides recommendations and/or alerts based upon biological markers within the physiological sample.

A sample analyzer 704 is provided to receive a physiological sample and to analyze the sample for one or more specific biological markers within the physiological sample. In some instances, the sample analyzer 704 is an oxidative stress tester, such as the Form Plus 3000 sold by Callegari SrI. In some instances, the sample analyzer 704 will output one or more numerical values associated with biological markers within the physiological sample. According to some embodiments, the sample analyzer 704 measures a blood sample for a pro-oxidant level (e.g. FORT assay), and may measure a blood sample for an anti-oxidant level (e.g. FORD assay). The methods and systems are not limited to use of a specific analyzer or manufacturer; neither are the systems and methods limited to particular assays, for example other assays other than FORD/FORT may be used. As improved and better ‘anti-oxidants’ assays are developed and emerge, they may be incorporated and applied to the systems and methods described. For example, for anti-oxidants, there is a reliable indication of ARH with glutathione. Additionally, anti-oxidant enzymes such as superoxide dismutase are reliable indicators of ARH and may used in place of or in combination with FORD. Similarly, examples of other pro-oxidants such as protein carbonyls and isoprostanes are reliable indicators of ARH and may used in place of or in combination with FORT.

The computing device 702 is equipped with one or more processors 706 and computer-readable storage media 708 to store one or more programs, applications, modules, data, and algorithms. The computer-readable storage media 708 is non-transitory and may store various instructions, routines, operations, and modules that, when executed, cause the processors to perform various activities. In some implementations, the one or more processors 706 are central processor units (CPU), graphics processing units (GPU) or both CPU and GPU, or any other sort of processing unit. The non-transitory computer-readable storage media 708 may include volatile and nonvolatile, removable and non-removable tangible, physical media implemented in technology for storage of information, such as computer readable instructions, data structures, program modules, or other data. System memory, removable storage, and non-removable storage are all examples of non-transitory computer-readable media. Non-transitory computer-readable storage media may include RAM, ROM, EEPROM, flash memory or other memory technology, CD-ROM, digital versatile disks (DVD) or other optical storage, magnetic cassettes, magnetic tape, magnetic disk storage or other magnetic storage devices, or any other tangible, physical medium which can be used to store the desired information and which can be accessed by the computing device 702.

The computer-readable storage media stores a contextual module 710, redox module 712, a rules module 714, and an alert module 716. Each of these modules may be able to access a database 718 as will be described further below.

The redox module 712 receives, as an input, results of the physiological sample analysis provided by the sample analyzer 704. In some instances, the input to the redox module 712 is a series of numerical values associated with biological markers based on colorimetric assays that detect physical reactions. In some cases, these numerical values relate to pro-oxidant values and anti-oxidant values. The redox module 712 may additionally generate a range of values for a subject, such as a threshold range indicating normal high and low values. The threshold values, which are described in further detail above, may be individualized to a subject based upon historical physiological samples.

The rules module 714 compares a specific sample result to the threshold values for the subject. The comparison may be accomplished by comparing a numerical value associated with the sample result to the threshold values stored for that subject.

The alert module 716 may provide feedback to the subject, or a clinician, and may indicate that a subject should take corrective action.

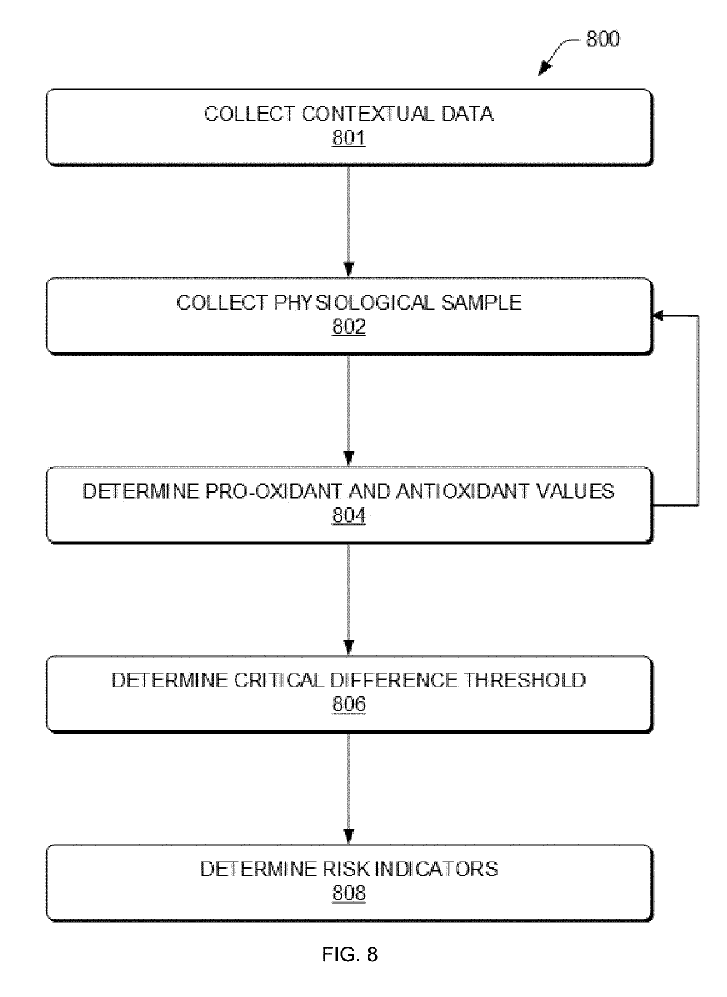

FIGS. 8, 9, and 10 are flow diagrams showing several illustrative routines for tracking redox homeostasis according to embodiments described herein. It should be appreciated that the logical operations described herein with respect to FIGS. 8, 9, and 10 are implemented (1) as a sequence of computer implemented acts or program modules running on a computing system and/or (2) as interconnected machine logic circuits or circuit modules within the computing system. The implementation is a matter of choice dependent on the performance and other requirements of the computing system. Accordingly, the logical operations described herein are referred to variously as operations, structural devices, acts, or modules. These operations, structural devices, acts, and modules may be implemented in software, in firmware, in special purpose digital logic, and any combination thereof. It should also be appreciated that more or fewer operations may be performed than shown in the figures and described herein. These operations may also be performed in parallel, or in a different order than those described herein, and may be performed by multiple devices. Of course, in some embodiments, the operations may be performed on a single device configured as described herein.

FIG. 8 illustrates an example process 800 for determining risk indicators for a subject. At block 801 contextual data is collected. At block 802, a physiological sample is collected and analyzed. At block 804, the pro-oxidant and anti-oxidant values are determined for the physiological sample. This may be performed, for example, by conducting the FORT and/or FORD assays as described herein. At block 806, a critical difference threshold is determined. At block 808, risk indicators are determined.

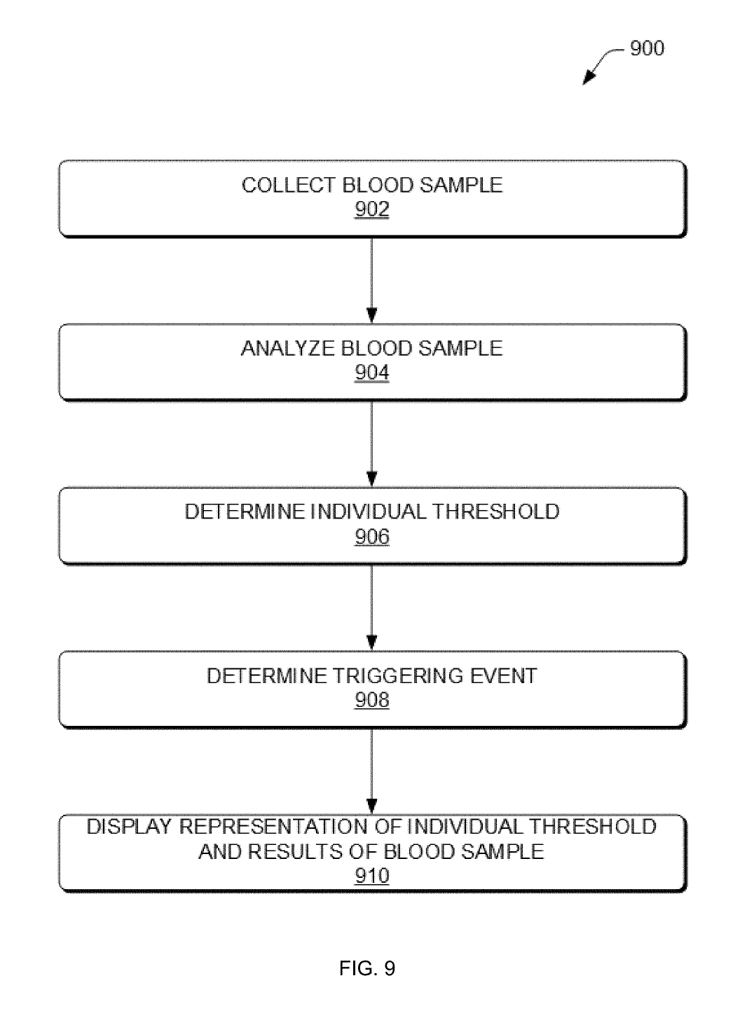

FIG. 9 illustrates another example process 900 for determining risk indicators for a subject. At block 902, a blood sample is collected. This may be through any suitable method and location; however, in some instances, the blood sample is one or more whole blood capillary samples which may be taken from each ear lobe. In some particular instances, 20 μl of blood is collected for a FORT assay and 50 μl of blood is collected for a FORD assay. At block 904, the blood sample is analyzed. In some cases, the blood samples are analyzed according to a FORT assay and a FORD assay to determine pro-oxidant values and anti-oxidant values in the sample.

At block 906, an individual threshold is determined. Initially, where there is not sufficient historical data for a subject, an initial threshold may be determined based upon a larger population, such as by using a Bayesian probability model as described herein, or any model or approach taken for generating reference ranges where the approach enables the reference ranges to adapt and be updated from data collected longitudinally and serially or in real-time, to determine a suitable range within which the pro-oxidant and antioxidant values should fall. However, as additional data becomes available from a subject, such as contextual data and blood sample data, an individualized threshold, which may be referred to as a critical difference threshold, can be determined. This may take into account the historical data for a subject, since a particular subject may have a differing range of normal values for the pro-oxidant values and anti-oxidant values than a larger population may exhibit. In addition, the range of values may change over time as the subject alters their diet, exercise regimen, and/or overall level of fitness and health. Accordingly, more recent samples may be given greater weight in determining the individual critical difference threshold.

At block 908, any triggering events are determined. The triggering events may be determined by identifying a pro-oxidant value or an anti-oxidant value that falls outside the critical difference threshold, such as being above a threshold maximum, or below a threshold minimum. The triggering events may be offset by any of the contextual data. For example, if the contextual data indicates that the subject is currently sick, the triggering event may be modified to a lower level of severity given the explanation for a value that falls outside the critical difference threshold.

At block 910, a representation, such as graphical representation, may be displayed that shows the individual threshold and the results of the most recent blood sample analysis. For example, the representation may be a visual representation, such as a graph, that is representative of a critical difference threshold and may also plot one or more values associated with blood sample assays. In some instances, the most recent blood sample analysis is plotted showing the results of the FORT and/or FORD assays. Of course, historical blood sample analyses may also be shown to allow a subject or clinician to view a historical trend in comparison with an individualized critical difference threshold.

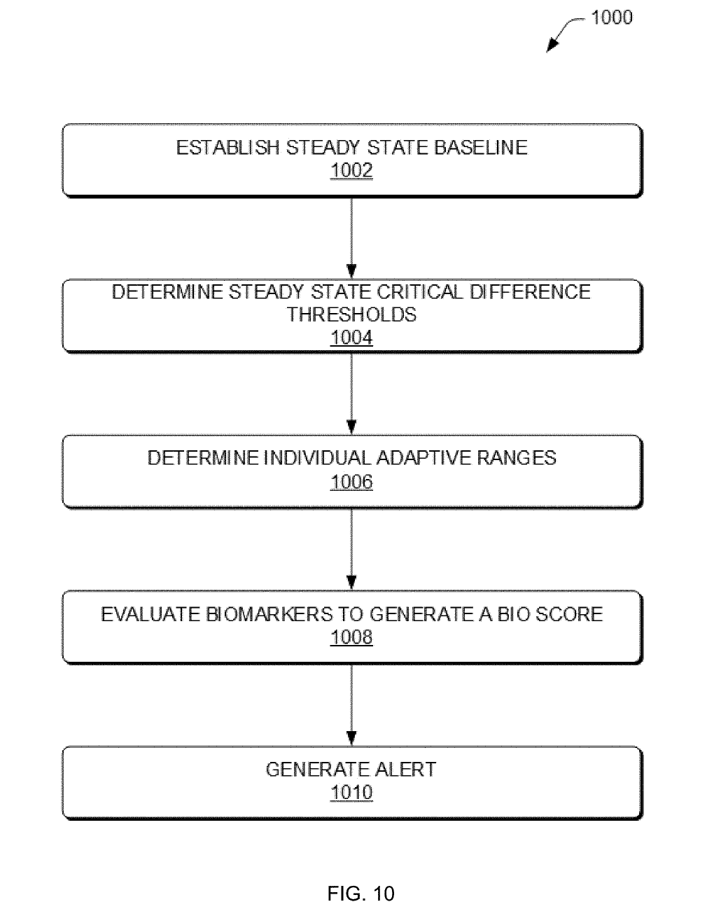

FIG. 10 is another example process 1000 for generating alerts for a subject. At block 1002, a steady state baseline is determined. This may be determined based upon a particular subject to be used as an input to calculate an individual critical difference threshold. In some cases, it is established by collecting one or more physiological samples from a subject that is healthy and in a fasted, hydrated, and rested state at the time of sample collection.

At block 1004, a steady state critical difference threshold is determined. The initial thresholds may be based upon the clinical range published by the manufacturer of the testing equipment. Additionally, the initial thresholds may be based upon prior collected physiological samples from a larger population of subjects. The larger population upon which the initial thresholds are based may be segmented to provide similarity to the subject. For example, if the subject is an elite athlete in a given sport, the sample population may be selected as also being elite athletes in the given sport. The steady state critical difference threshold is determined to indicate a range of normal values for the individual subject.

At block 1006, individual critical difference values are determined. This may be accomplished, for example, by using historical physiological sample analysis results for a given subject to determine normal ranges. This may also take into consideration the contextual data for a given subject, such as a subject's current health, recent exercise regimens, and diet, among other things. The individual adaptive ranges are the individual critical difference thresholds for a given subject.

At block 1008, biomarkers are evaluated to generate a bio score. For example, one or more blood samples may be used to determine anti-oxidant and pro-oxidant levels, which may then be used as a bio score, or may be used in combination with other data to generate the bio score.

At block 1010, an alert is generated. The alert may be based upon the bio score exceeding a threshold maximum. In some instances, the alert may be based upon the bio score being less than a threshold minimum. In some instances, the alert may be based upon the bio score being near or close to the threshold maximum and/or threshold minimum. In some cases, the threshold maximum and the threshold minimum are determined by the individual adaptive ranges. The alert may indicate that the bio score is outside the individual adaptive range. The alert may additionally provide suggestions for ameliorating the condition that caused the alert to be generated. For example, the alert may indicate that the subject should get more sleep, change their diet, or alter their workout intensity, among other things. The alert may additionally indicate a severity of the condition. For example, if a pro-oxidant value is above the maximum threshold for the pro-oxidant individual adaptive range maximum, by 5%, the alert may indicate a minor severity and suggest a less aggressive measure to ameliorate the condition. If, however, the pro-oxidant value is 25% or more above the maximum, the alert may indicate a very severe condition and suggest aggressive actions to remediate the condition.

While the preceding discussion describes exemplary systems and methods to provide feedback based on the assessments, other avenues for provision of results and/or interventions may also be used in place of or in addition to the methods described up to this point. For example, feedback could be provided in oral form, in person or through an auditory communication device, through text, via, for example text messaging, or any other appropriate communication method.

### Exemplary Embodiments

1. A computer readable storage media storing instructions that, when executed by one or more processors, cause the one or more processors to perform acts including: retrieving, from a database, first data indicative of historical biomarkers; determining based upon the first data, a critical value range, the critical value range defined at least in part by a maximum threshold for the biomarkers and a minimum threshold for the biomarkers; receiving, from a physiological sample assay device, a test sample value collected from a subject; determining that the test sample value is outside the critical value range; detecting, based at least in part upon the test sample value being outside the critical value range, that the subject is at increased risk for injury and illness; determining an intervention plan for the subject to reduce the likelihood of injury and illness; and displaying, on a display device, the intervention plan.
2. The computer readable storage media of embodiment 1, wherein the biomarkers include values for pro-oxidant and anti-oxidant markers.
3. The computer readable storage media as of embodiments 1 or 2, wherein the biological sample assay device includes a redox analyzer.
4. The computer readable storage media as of any of embodiments 1-3, wherein the test sample includes a blood sample.
5. The computer readable storage media of embodiment 4, wherein the blood sample includes a whole blood capillary sample.
6. The computer readable storage media of any of embodiments 1-5, wherein the critical value range is based on at least two critical difference thresholds determined for an individual subject, the critical difference thresholds based at least in part upon historical test sample values collected over a period of time for the subject.
7. The computer readable storage media of in embodiment 6, wherein more recent test samples are given a higher weighting in determining the critical difference value range.
8. The computer readable storage media of embodiments 6 or 7, wherein the acts further include displaying, on the display device, a visual representation of the critical difference thresholds, the critical difference range, and a new test sample value.
9. A system, including: a redox analyzer; and a computing device having: one or more processors; memory coupled to the one or more processors, the memory storing instructions that, when executed, cause the one or more processors to: receive, from the redox analyzer, first values associated with a physiological sample from a first subject, the first values indicating pro-oxidant and anti-oxidant values associated with the physiological sample; retrieve, from a database, historical values associated with historical physiological samples; determine, based at least in part upon the historical values, a critical difference threshold for the subject; determine, based upon comparing the first values with the critical difference threshold, that one or more of the first values are outside a range defined by the critical difference threshold; detect, based at least in part upon the one or more of the first values being outside the range, that the first subject is at increased risk of injury and illness; generate an alert indicating that the first subject is at increased risk of injury and illness; and present, on a display device associated with the computing device, an indication of the alert and a recommended action plan.
10. The system of embodiment 9, wherein the redox analyzer and the computing device are contained within a common housing.
11. The system of embodiment 9 or 10, wherein the instructions further cause the one or more processors to present, on the display device, a visual indication of the critical difference threshold and the first values.
12. The system of any of embodiments 9-11, wherein the critical difference threshold is determined, at least in part, on a Bayesian predictive model, or any approach taken for generating reference ranges where the approach enables the reference ranges to adapt and be updated from data collected longitudinally and serially or in real-time.
13. The system of any of embodiments 9-12, wherein the redox analyzer provides a FORD value and a FORT value.
14. The system of any of embodiments 9-13, wherein the recommended action plan includes modifying one or more of a diet, an exercise regimen, an exercise intensity, or a sleep pattern of the subject.
15. The system of any of embodiments 9-14, wherein the indication of the alert includes a colored light.
16. The system of any of embodiments 9-15, wherein the instructions further cause the one or more processors to update the database with the first values and determine, based at least in part upon the historical values and the first values, a new critical difference threshold.
17. The system of any of embodiments 9-16, wherein the instructions further cause the one or more processors to receive contextual data associated with the subject, the contextual data indicating one or more of dietary habits, rest habits, current mood, energy levels, or injury status, illness, wellness, muscle soreness of the subject.
18. The system of embodiment 17, wherein the first values are modified by the contextual data.
19. A method of reducing the risk of injury and illness in a team of elite athletes including: obtaining contextual data from each individual on the team; obtaining at least five physiological samples from each individual on the team wherein the obtaining of each sample from an individual is separated by at least one week; detecting pro-oxidant and anti-oxidant markers in the physiological samples using at least two colorimetric assays; inputting values based on the detecting into a system; calculating a critical difference threshold for each individual on the team; obtaining additional physiological samples from each individual on the team; detecting pro-oxidant and anti-oxidant markers in the additional physiological samples using at least two colorimetric assays; determining whether test values based on the detecting are within an individual's critical difference threshold; identifying a team member as at increased risk for injury and illness if the individual's test values are outside of the individual's critical difference threshold; and implementing an intervention for each team member identified as at-risk thereby reducing the risk of injury and illness in a team of elite athletes.
20. A method of detecting an increased risk of injury or illness in a subject including: obtaining at least four physiological samples obtained from the subject at different time points; assaying each sample for FORD and FORT values; comparing the FORD and FORT values to a critical difference range established for the subject; detecting an increased risk of injury or illness in the subject if the FORD and/or FORT value falls outside of the critical difference range.
21. Use of a portable redox analyzer to detect an increased risk of injury or illness in a subject including: obtaining a sample from the subject; assaying the sample for FORD and FORT values; comparing the FORD and FORT values to a critical difference range established for the subject; detecting an increased risk of injury or illness in the subject if the FORD and/or FORT value falls outside of the critical difference range.
22. A method of establishing a critical difference range for a subject including: obtaining at least 4 physiological samples from the subject wherein each physiological sample is obtained at within a desired time-frame, for example at least 24 hours apart or at least 48 hours apart, assaying each sample for FORD and FORT values; calculating an upper threshold for pro-oxidant values; calculating a lower threshold for anti-oxidant values; determining the difference between the upper threshold and the lower threshold, thereby establishing a critical difference range for the subject.
23. A method of embodiment 22 including using the established critical difference range to detect an increased risk of injury or illness in the subject.
24. A method of embodiment 22 or 23 including obtaining an additional physiological sample from the subject; assaying the additional physiological sample for FORD and FORT values; detecting an increased risk of injury or illness in the subject if the FORD and/or FORT value falls outside of the critical difference range.
25. Any of the preceding embodiments including use of glutathione and/or superoxide dismutase levels to determine anti-oxidant scores.
26. Any of the preceding embodiments including use of protein carbonyl and isoprostanes levels to determine pro-oxidants scores.
27. Use of the systems and methods disclosed herein to identify the health status of an individual and identifying and recommending appropriate interventions and actions to help maintain health and wellness.
28. A use of embodiment 27 wherein maintenance of health and wellness is identified based on maintenance of pro-oxidant and anti-oxidant levels with the individual's critical difference range.
29. A use of claim 27 wherein maintenance of health and wellness is identified by continued absence of illness and injury.
30. Use of the systems and methods disclosed herein to identify the health status and wellness of an individual and identifying and recommending appropriate interventions and actions to restore, maintain, or sustain health and wellness.

### Example 1

(1) Contextual data on a subject (e.g., athlete) is gathered via questionnaire prior to testing (see, e.g., FIGS. 3A-3E);

(2) Whole blood capillary samples are taken from each ear lobe (20 μl for FORT and 50 μl for FORD);

(3) Samples are immediately processed and analyzed at room temperature in line with the instructions provided by the analyzer's manufacturer (e.g., a Callegari analyzer, The Catellani Group)

(4) Results are manually uploaded to App by tester;

(5) R code is called up within the redox App, the relevant arguments processed by the algorithm and the updated individual threshold returned;

(6) Rules engine determines (a) the status of the values returned, (b) whether an alert is to be triggered, (c) which alert to trigger;

(7) Result and associated alert are displayed in App (see, for example, FIG. 4B). The alert warns the client (e.g., coach, doctor) when a subject (e.g., athlete, patient) is close to or outside his/her individual thresholds (indicating the need to manage the training load and recovery requirements of the subject). It lists the potential outcomes and offers recommendations on how to improve the subject's values;

(8) Scientists can also log in and provide additional feedback to clients on results via the App.

### Example 2

Background & Overview. A balance (redox homeostasis) between pro- and anti-oxidants is essential for muscle function and training adaptation. There is a relationship between alterations in redox homeostasis (ARH) and performance, injury, and illness in athletes and ARH often leads to maladaptation and fatigue. ARH occurs across the season in athletes, with marked variation around intensified training phases and in athletes competing. By using a global standard (FORT and FORD assays) to test regularly for ARH, individual tolerance to training loads is identified as are risk indicators for injury and illness.

Disclosed in this Example is a reliable method of tracking and analyzing stress and defense levels and presenting these against an individualized baseline and threshold to indicate risk of injury and fatigue. Here, the method is described as a service whereby a Sports Scientist (SS) collects ‘Redox’ samples via lancet using a Callegari analyzer and consumables every 2 weeks for a period of 12 weeks i.e. 6 tests. The analyzer produces a Stress (FORT) and Defense (FORD) result for each sample.

The SS enters the values into the application for Stress (Fort) and Defense (Ford). The athletes will answer a questionnaire to give additional contextual information to the values. (See FIGS. 3A-3E). The application will run an algorithm to produce an individualized critical difference threshold for Stress and Defense and provide a traffic light indicator of risk for each value. The Indicator types are:
Red=critical intervention suggested
Amber=intervention suggested
Green=good profile.
   Other color, dial, numeric or other scales may also be used to indicate the risk level.

The application will automatically show the current traffic light indicator for each athlete for Stress and Defense and will provide advice content for each athlete (See FIGS. 4B and 6). Additionally, the SS can review and comment on the athlete profiles.

In this Example, the process is quick (e.g., 10-15 min per athlete). The system automatically provides a CD threshold, identifies athletes at risk and provides alerts and recommendations (See FIGS. 4B and 6). The system also allows viewing of athletes who are at risk of illness or injury; current and past stress and defense levels against individual critical difference thresholds; current and past contextual information and notes for each result; current and past comments from SS (where applicable); advice to improve stress and defense levels.

In this Example, for calculating the critical difference value, a result contains both a Stress and Defense value. An athlete must have a total of 3 steady state results before a CD threshold can be applied. A result is not included in the calculation if the athlete has had any of the following in the week prior to testing: bacterial or viral illness; injury; or a high fatigue level.

The CD calculation will commence once the fourth result is entered, (i.e the first 3 results are not plotted). A rolling mean is applied using a first in first out (fifo) queue method containing a maximum of seven results. The most recent result is not included in the CD calculation. A user (e.g., SS) must determine if a set of results should be excluded as the criteria can be somewhat subjective. Thus, in this Example, a user (e.g., SS) will have the ability to include/exclude a result. Until a steady state is determined, the system will use a standard range for all athletes:
Stress—green=1.22-2.0, amber=2.0-2.5, red=2.5+
Defense—green=1.20-2.0, amber=1.1-1.20, red=>1.1

### Example 3

The Aim of Example 1 was to: 1) assess the repeatability of the FFT at rest in elite athletes, and 2) calculate the analytical variation, BV, CDV, and index of individuality for FFT, RBC GSH, lutein, and α and γ-tocopherol in well-trained participants.

Methods. Part 1: Repeatability: Following institutional ethical approval, 15 national and internationally ranked (includes Olympic and world medalists) endurance athletes (n=8 males and 7 females; age (mean±SD) 22±4 y; body mass 72±7.1 kg; height 1.79±0.03 m; \dot{V}O₂ₘₐₓ 66±6.5 ml·kg⁻¹·min⁻¹) volunteered to participate. Athletes were free living and attending a national training centre, not taking any medications, and were subject to United Kingdom Anti-doping controls and testing procedures. All athletes were tested in the general preparation phase of the annual cycle and were following nutritional guidelines administered via the English Institute of Sport system. Testing was carried out between 8 a.m. and 9 a.m. in a fasted, hydrated and rested state. The following tests were performed after informed consent was obtained as a part of sports science support provision, with procedures approved by the Internal Review Board of the English Institute of Sport. Written informed consent being obtained from the athletes.

Blood sampling. Whole blood capillary samples (50 μL for FORD, and 20 μL for FORT) were taken from each ear lobe (duplicate samples), processed and analyzed immediately at room temperature in line with the manufacturer's instructions (Callegari SpA, Catellani Group, Parma, Italy). Briefly, heparinized capillary samples are immediately mixed with the reagent, centrifuged and analyzed colorimetrically (CR3000, Callegari SpA, Catellani Group, Parma, Italy).

Biochemical analysis. The FORT assay and FORD assay are described previously.

Part 2: Biological Variation: The ethics committee of St Mary's University approved the study. Twelve well-trained male participants (n=12) were recruited (age (mean±SD) 30±7 y; weight 81.9±8.2 kg; height 1.86±0.1 m). All participants provided written, informed consent following completion of a health questionnaire. Healthy participants were selected based on recommendations for performing studies on BV [Fraser, Biological variation: from principles to practice. AACC Press. Washington, D.C.; 2001]. Strict control of environmental factors was undertaken to reduce variability and control for factors known to disrupt redox homeostasis e.g. exercise [Mullins et al., Biomarkers. 2013; 18: 446-454], high fat meal [Bloomer et al., Lipids Health Dis. 2010; 9: 79], infection [Schwarz, Free Radical Biology and Medicine. 1996; 21: 641-649], metabolic disease [Le Lay et al., Andriantsitohaina R. Oxidative Stress and Metabolic Pathologies—From an Adipocentric Point of View. Oxidative Medicine and Cellular Longevity. Hindawi Publishing Corporation; 2014; 1-18], anti-oxidant supplements [Ristow et al., Proc Natl Acad Sci USA. 2009; 106: 8665-8670]. All participants abstained from physical exercise, alcohol consumption for 72 hours prior to testing, caffeine for 24 hours, and maintained their normal dietary habits the day before testing.

Participants arrived at the laboratory at 7.30 a.m. Venous and capillary blood samples were collected every two hours through the day at 0800, 1000, 1200, 1400, 1600, 1800 for the analysis of RBC GSH, α and γ tocopherol, lutein, and the FORT and FORD assay. To minimize sources of variation the following criteria were applied; fasted on arrival to the laboratory and remaining without food until after the last blood sample was collected at 1800 to control for hormonal fluctuations; water was allowed ad libitum throughout only, remaining supine on an examination couch at a comfortable temperature throughout (20-22° C.) for a minimum of 20 minutes prior to each blood draw.

Blood sampling. Blood was sampled at the antecubital vein for a total of six blood draws over 10 hours. For the RBC GSH a single 5 ml sample of venous blood was collected in a lithium heparin vacutainer tube (BD system; New Jersey, USA), and for lutein, α and γ tocopherol, a single 5 ml sample of venous blood collected in a serum separator (SST) vacutainer tube (BD system; New Jersey, USA). For details on the blood sampling of FFT see part 1 (above). For RBC GSH, lutein, α and γ tocopherol, assays the samples were centrifuged and aliquots were stored at −50° C. for later analysis. To minimize analytical variance, RBC GSH, lutein, α and γ-tocopherol were analyzed under the same analytical conditions; the same batch of reagents and standards, and by the same laboratory biochemists using the same analyzers.

Biochemical Analysis. Serum α-tocopherol, γ-gamma tocopherol and serum lutein were measured by reverse-phase high-pressure liquid chromatography (HPLC) on an Agilent 1200 series system (Agilent, Manchester, U.K) with ultra-violet/visible detection using a modification of the method of Thurnham et al., Clinical Chemistry. 1988; 34: 377-381. Samples were protected from light on analysis to reduce oxidation, and serum separated for analysis by centrifugation at 3000 rpm for 10 minutes. Calibration was carried out using pure tocopherol standards (Sigma chemical Co, Poole, Dorset, U.K) dissolved in ethanol and a lutein standard (AASC Ltd, Southampton, U.K.) dissolved in hexane/chloroform, in which concentrations were derived by scanning ultra-violet spectrophotometry and applying the molar extinction coefficients for each substance. Quantification involved internal standardization and dose-response curves established with authentic standards. Serum α-tocopherol, γ-gamma tocopherol and serum lutein were reported as μmol·L⁻¹. Intra assay CV were 3.3%, 6.8%, and 3.5% for α-tocopherol, γ-tocopherol and lutein respectively.

RBC GSH was measured by the method of Beutler et al., J Lab Clin Med. 1963; 61: 882-888 using the chromogenic reaction of 5,5-dithiobis-(2-nitrobenzoic acid) (DTNB) with sulphydryl groups. The millimolar extinction coefficient of the DTNB anion was applied to derive concentrations of GSH in whole blood and the erythrocyte GSH was calculated using the haematocrit (packed cell volume) of the blood sample. RBC GSH reported as mmol GSH per litre of red cells, with the intra-assay CV for RBC GSH of 2.4%.

Statistical Analysis. Numerical (mean±standard deviation) and graphical summaries (case profile plots) were provided for each biomarker over time. In addition, plots of the relative change from baseline (%) were generated for each variable. There was no evidence against normality for the distributions of each biomarker at each time point. As each response variable of interest (i.e. the set of six biomarkers) is a continuous variable, a linear mixed model was used to model the change over time. A random effect for each individual was incorporated in all models and the within-individual correlation over time was specified as unstructured. The time when the testing was recorded was modeled as a fixed effect, initially as a categorical variable in order to allow a comparison in the mean change at each time point and then as a continuous variable in order to compare the slopes for each biomarker over time. Relationships between biomarker variables were examined using Pearson correlations. All statistical analyses were carried out using R (version 3.1) and the nlme package. The significance level was set at alpha=0.05. Model assumptions were visually assessed for each response at each time point using residual plots from the fitted model.

Part 1: Analytical variation (CV_{A}). The analytical coefficient of variation (CV_{A}), the intra-assay CV_{A} (%) were calculated for the FORT and FORD test using methodology of Fraser and Harris, Biological variation: from principles to practice. AACC Press. Washington, D.C.; 2001. CV_{A} is calculated using the following formula:

$${CV}_{A} = \frac{SD}{X} \times 100 ( \% ) \qquad Eq . 1$$

Where X=mean and SD=standard deviation.

Part 2: Analytical and Biological Variation. The CV_{A} for RBC GSH, lutein, and α and γ-tocopherols were calculated using the formula above, and derived from duplicate samples. The within subject biological variation (CV_{w}), between subject variation (CV_{B}), CDV, and index of individuality (II) were calculated according to methods of Fraser and Harris, Biological variation: from principles to practice. AACC Press. Washington, D.C.; 2001; Fraser C G. Reference change values. Clinical Chemistry and Laboratory Medicine. 50. doi:10.1515/cclm.2011.733. A missing value was substituted by the mean value for that participant in the analysis. CDV was calculated using the following formula:

CDV=2^{1/2}·Z·(CV_{A}²+CV_{W}²)^{1/2}

Given duplicate samples were run on RBC GSH, lutein, and α and γ-tocopherols, the CDV was adjusted to the following, where by n₂ refers to the number of analytical replicates (duplicates):

CDV for duplicate analysis=2^{1/2}·Z·(CV_{A}²/n₂+CV_{W}²)^{1/2}

II was calculated using the following formula:

II=(CVa²+CV_{w}²)^{1/2}/CV_{B}

Results. Repeatability of FFT (part 1). The repeatability of the FORT and FORD assay was 3.9% and 3.7% respectively. The FORT and FORD results for the squad were 1.66 (0.36) mmol·L⁻¹ and 1.76 (0.17) mmol·L⁻¹ respectively.

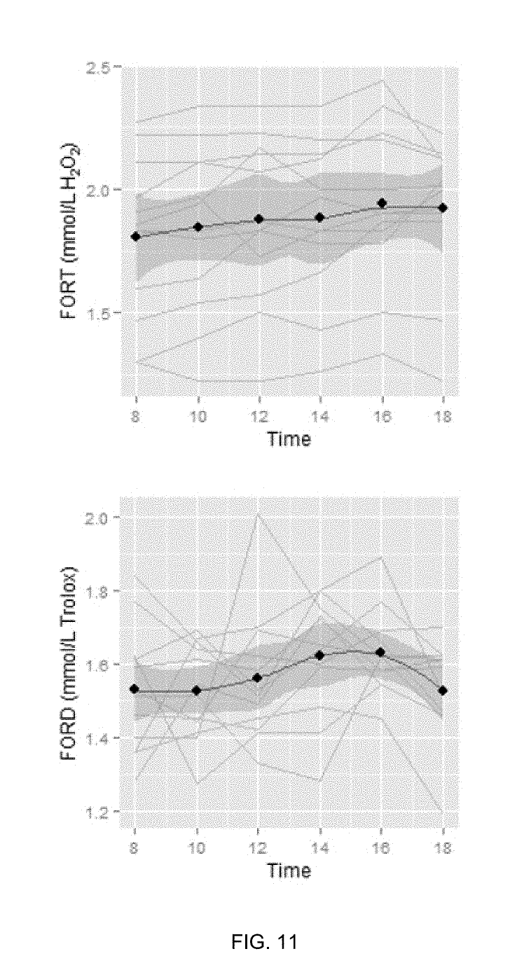

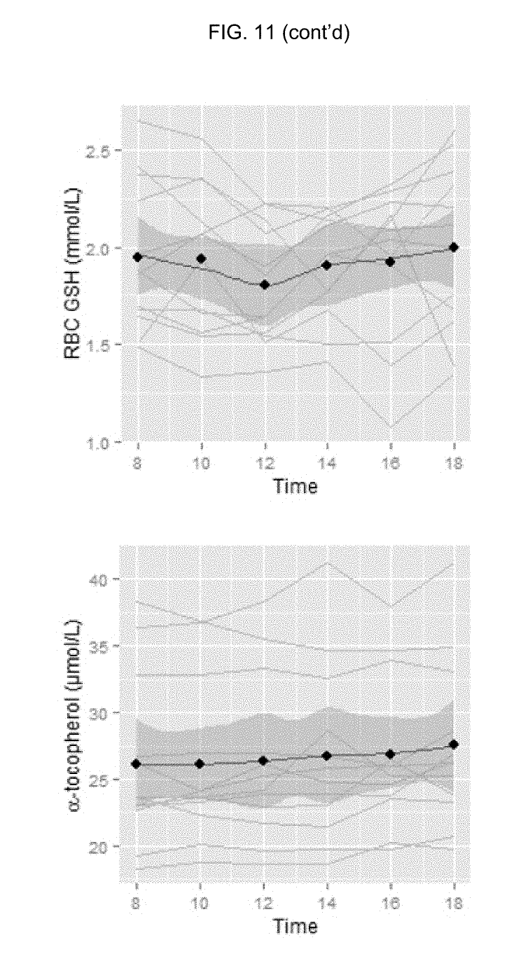

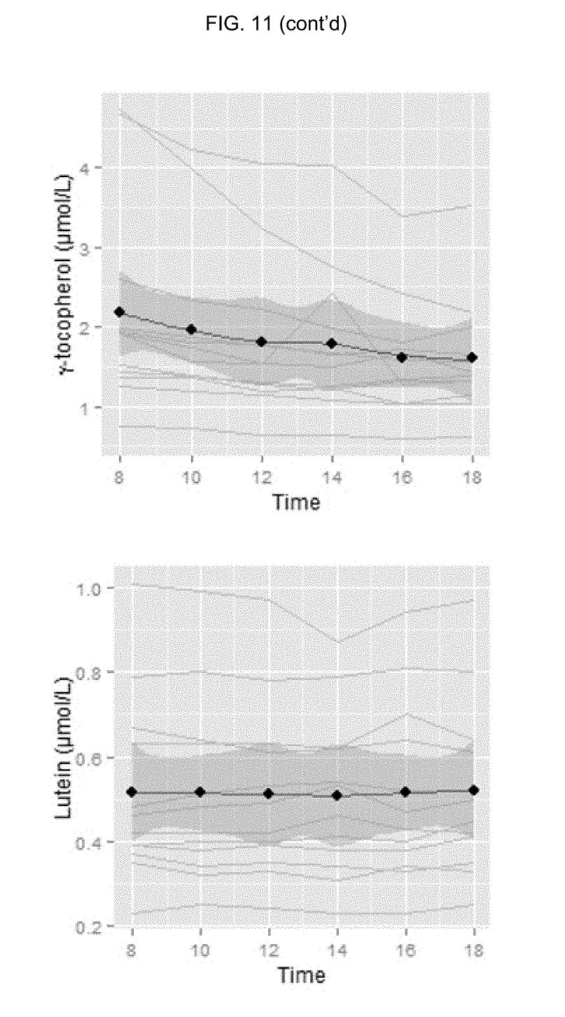

Analytical, BV, CDV and index of individuality for the FFT (part 2). A significant effect for time was observed for FORT (p<0.001), γ-tocopherol (p<0.001) and α-tocopherol (p=0.002) over the 10-hours, indicating circadian variation (FIG. 11).

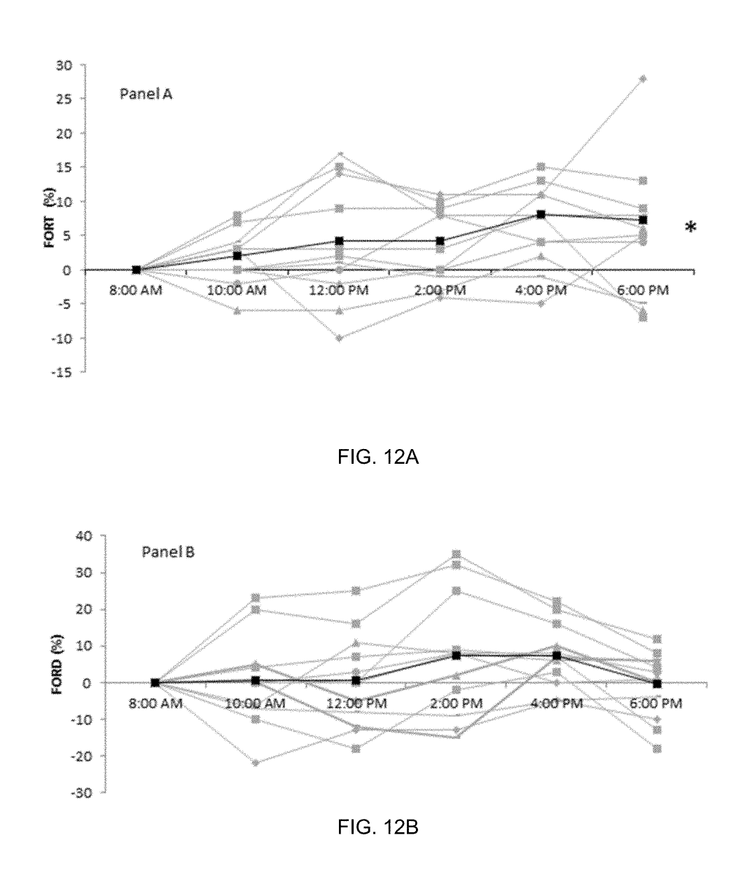

There was no effect for time for lutein (p=0.60), RBC GSH (p=0.52) and FORD (p=0.26). FIGS. 12A and 12B show the temporal effect for FORT and FORD, α- and γ-tocopherols respectively, expressed as the relative change from baseline.

Table 3 summarizes the mean (SD) for the FORT, FORD, RBC GSH, lutein and α and γ-tocopherols.

TABLE 3
Absolute mean ± SD concentrations for FORT, FORD,
RBC GSH, lutein, and α and γ-tocopherols

| | 8 am | 10 am | 12 pm | 2 pm | 4 pm | 6 pm |
|---|---|---|---|---|---|---|
| FORT (mmol · L⁻¹ | 1.81 | 1.84 | 1.88 | 1.88 | 1.94 | 1.92 |
| H₂O₂) | (0.33) | (0.34) | (0.33) | (0.32) | (0.33) | (0.29) |
| FORD (mmol · L⁻¹ | 1.53 | 1.52 | 1.56 | 1.62 | 1.63 | 1.52 |
| Trolox) | (0.17) | (0.13) | (0.18) | (0.16) | (0.12) | (0.13) |
| γ-tocopherol (μmol · L⁻¹) | 2.17 | 1.97 | 1.81 | 1.79 | 1.60 | 1.60 |
| | (1.26) | (1.08) | (0.96) | (0.92) | (0.73) | (0.73) |
| α-tocopherol (μmol · L⁻¹) | 26.2 | 26.1 | 26.4 | 26.8 | 26.8 | 27.5 |
| | (6.4) | (6.1) | (6.2) | (6.6) | (5.7) | (6.2) |
| Lutein (μmol · L⁻¹) | 0.52 | 0.51 | 0.51 | 0.51 | 0.52 | 0.52 |
| | (0.22) | (0.22) | (0.21) | (0.19) | (0.21) | (0.21) |
| RBC GSH (mmol · L⁻¹) | 1.95 | 1.94 | 1.80 | 1.91 | 1.92 | 2.00 |
| | (0.39) | (0.38) | (0.30) | (0.28) | (0.39) | (0.43) |

Table 4 summarizes the analytical and biological variability, CDV and index of individuality for FORT, FORD, RBC GSH, lutein and α and γ-tocopherols. The analytical variability (CV_{A}%) for all the biomarkers indicates good precision for the assays (Table 4). The biomarker displaying the greatest analytical (6.8%), biological (12.5%), and between subject variability (51.4%), and CDV (37%) was γ-tocopherol.

TABLE 4
Analytical and biological variation, critical difference
values and index of individuality for FORT, FORD,
RBC GSH, lutein, and α and γ-tocopherols

| Biomarker | CV_{A} % | CV_{W} % | CV_{B} % | II | CDV % |
|---|---|---|---|---|---|
| FORT | 3.9 | 5.0 | 17.3 | 0.29 | 17.4 |
| FORD | 3.7 | 7.5 | 9.6 | 0.78 | 23.8 |
| γ-tocopherol | 6.8 | 12.5 | 51.4 | 0.24 | 37.0 |
| α-tocopherol | 3.3 | 4.5 | 23.4 | 0.19 | 14.0 |
| Lutein | 3.5 | 3.9 | 40.8 | 0.10 | 12.8 |
| RBC GSH | 2.4 | 9.6 | 18.9 | 0.51 | 26.9 |

CV_{A} % = analytical variation
CV_{W} % = within subject
CV_{B} % = between subject variation
II = index of individuality
CDV % = critical difference value

For the redox biomarker FORT, moderate to weak relationships were observed for FORT and RBC GSH (r=−0.41; p<0.001), α-tocopherol (r=0.47; p<0.001) and lutein (r=−0.24; p=0.04) respectively. For plasma FORD, a strong relationship with RBC GSH was observed for AM measures only (p=0.001; r=0.62). For biomarkers of nutritional status; strong to moderate relationships were observed for α-tocopherol and γ-tocopherol (r=0.78; p<0.001); α-tocopherol and lutein (r=0.37; p=0.001); γ-tocopherol and lutein (r=0.41; p<0.001); and a weak correlation with RBC GSH and γ-tocopherol (r=0.23; p=0.04).

Discussion New information on the BV and accompanying CDV and index of individuality (II) for the OS and nutritional biomarkers FORT, FORD, RBC GSH, lutein and γ-tocopherol is provided. Furthermore, evidence of a significant circadian effect for FORT, and serum α and γ-tocopherol is provided; no effect was observed for the anti-oxidants lutein, FORD or RBC GSH. A circadian effect for FORT and γ-tocopherol represents a finding not reported elsewhere. The repeatability (CV_{A}) of the POC test for both the FORT (3.9%) and the FORD assay (3.7%), are comparable with reported laboratory measures of OS and are of sufficient analytical precision to be used clinically. Indeed, the CV_{A} for FORT shows better precision than that reported for malondialdehyde (MDA) (6.2%), lipid hydroperoxides (4.6%) plasma isoprostanes (4.5%), and protein carbonyls (11.9%) in BV OS research [Davison et al., J Physiol Biochem. 2012; 68: 377-384; Mullins et al., Biomarkers. 2013; 18: 446-454; Dahwa et al., Biomarkers. 2014; 19: 154-158]. The CDV values vary between biomarkers, with large relative changes required for RBC GSH (>27%), FORD (24%) and γ-tocopherol (37%) before physiological significance could be confidently stated. For all OS and nutritional biomarkers reported here, the II indicates that reference ranges are of limited use in assessing meaningful changes in serial results in individuals. Interestingly, the participant with the largest relative increase in FORT (28% over the 10 hours), greater than the FORT RCV of 17% and thus deemed to be of physiological significance, had the lowest concentrations of the dietary anti-oxidants, γ-tocopherol, α-tocopherol, and lutein.

Proposed theoretical mechanisms for the observed elevations in FORT (OS) are: 1) elevations in FFA oxidation as a result of increased FFA's in keeping with a 24 hour fast and 2) coupled with reduced ATP demand-substrate oxidation (supine for 12 hours) and thus elevations in reducing equivalents (nicotinamide adenine dinucleotide; NADH₂ and flavin adenine dinucleotide; FADH₂) leading to increased mitochondrial superoxide and H₂O₂ formation and leak, ultimately elevating the basal levels of hydroperoxides. The generation of reducing equivalents being high during fatty acid metabolism, even at low physiological FFA concentrations [Seifert et al., Journal of Biological Chemistry. 2010; 285: 5748-5758] and the rate of mitochondrial H₂O₂ emission is increased when transitioning from carbohydrate to a high fat diet [Anderson et al., J Clin Invest. 2009; 119: 573-581]. In addition, a strong relationship exists between plasma FFA's and mitochondrial H₂O₂ production [Sahlin et al., Journal of Applied Physiology. 2010; 108: 780-787], and a 10-hour fast leads to persistent reductions in cytosolic GSH/GSSG [Anderson et al., J Clin Invest. 2009; 119: 573-581]. A significant inverse relationship between plasma FORT and RBC GSH concentrations was observed.

The results demonstrate that laboratory reference ranges are not useful for the interpretation of OS biomarkers when applied to serial results in individuals, on the basis none of the biomarkers demonstrated an index of individuality greater than 0.8, with a low of 0.10 for lutein, and a high of 0.78 for the FORD assay. An index of greater than 1.4 indicates results can be evaluated usefully against reference ranges [Fraser, Biological variation: from principles to practice. AACC Press. Washington, D.C.; 2001].

The strengths of this study are the level of methodological pre-analytical control applied to the participants. Such rigorous controls are a necessary feature of studies on BV to minimize sources of variability.

In this Example, the FFT, which includes a measure of plasma anti-oxidant capacity (FORD) was chosen for investigation because: 1) the FORD assay decreases in diseases of OS and is thus sensitive to depicting changes in OS; 2) uric acid is not a major component of the anti-oxidant activity of the assay; 3) measures of TAC have been shown to decrease with psychological stress, altitude, intense competition, training load and fatigued states [Lewis et al., Sports Med. 2015; 45: 379-409; Sivonova et al., Stress. 2004; 7: 183-188]; 4) GSH is reported to contribute to the anti-oxidant activity of the assay, and thus explain a significant proportion of the variability in the assay. For plasma FORD, a significant strong relationship with RBC GSH was observed for a.m. only.

### Example 4

Despite the huge body of research in the field of redox biology, few studies describing the redox responses to exercise in female athletes have been published (Lewis, et al., 2015. Sports Med 45: 379-409). In sub-elite and recreationally trained athletes, gender differences exist for ARH, with females typically exhibiting lower (OS) compared with males in lipid peroxidation measures Bloomer & Fisher-Wellman, 2008. Gend Med 5: 218-228).

An exercise challenge has been used consistently as a valid means of assessing redox responses and OS in the following groups or settings: young and old untrained and trained participants (Cobley, et al., 2014. Free Radical Biology and Medicine 70: 23-32), healthy and fatigued athletes (Tanskanen, et al., 2010. Journal of Sports Sciences 28: 309-317), and in response to environmental challenges (i.e. simulated altitude; Debevec, et al., 2014. Medicine & Science in Sports & Exercise: 46: 33.41). A maximal exercise challenge to push the athletes to exhaustion was chosen because (i) elite athletes are well adapted to their exercise modality and (ii) it would provide the ability to assess the redox response in elite healthy athletes (i.e. not in fatigued, overreached or over-trained athletes) to generate normative OS data (i.e. FORT and FORD) for elite healthy endurance athletes in response to exercise.

The aims of the current study were to, for the first time, (i) assess for ARH in response to sub-maximal and maximal exercise using the FFT, in elite non-fatigued endurance athletes, further validating the FFT in elite sport, and (ii) assess using the FFT whether differences in ARH exist between elite males and elite females.

Materials and methods. Subjects. Elite endurance athletes were recruited to the study; see Table 3 for athlete characteristics (female n=7; male n=15). The group was made up of elite runners and triathletes including Olympic finalists, European and Commonwealth medalists from distances of 400 m to marathon, and a European Ironman triathlon champion. All athletes provided written informed consent and completed a health questionnaire. Testing was carried out between December and September and the athletes described themselves as free from injury, illness, and under performance; most of the participants were tested in the competition preparation phase (May-August). The ethics committee of St Mary's University approved the study.

Experimental protocol. On the day of the test, the athletes arrived in the laboratory between 0700 and 0900 hours. They were well hydrated and had been instructed to undertake only light exercise during the previous 24 hours (classified as an “easy” aerobic session) and to abstain from high-intensity and resistance exercise during the previous 72 hours. Following completion of the informed consent and medical questionnaire, the participants were allowed to consume a standard breakfast (see ‘Diet’ section). 1.5-2 hours after ingestion of the standard breakfast, the athletes entered the exercise phase.

Diet. To control for the effect of the various breakfast choices on redox balance (RB), athletes were instructed to arrive fasted, having consumed a maximum of 500 ml of water only on waking. A standard high carbohydrate and protein breakfast was provided on arrival, in the form of a formulated high-energy sports nutrition bar (Powerbar Energise, Nestle Powerbar U.K.) and 500 ml milk shake (For Goodness Shakes, U.K.), thus ensuring no recognized sources of anti-oxidants (e.g. testing e.g. fruits, vegetables, high fibre cereal grains, seeds and nuts) were consumed immediately before the testing. Moreover, no tea, coffee or fruit juices were allowed. Water was allowed ad libitum. The athletes were instructed not to take any vitamin or mineral, or sports nutrition products (e.g. vitamin C tablets, iron) during the 24 hours before the testing or on the morning of the test. In addition, they were required to maintain their normal diet, and avoid unusual consumption of caffeinated drinks and foods and the consumption of alcohol in the 24 hours prior to testing.

Sub-maximal and maximal exercise protocol. After a 10 min warm-up on a motorised treadmill (Woodway E L G, Woodway USA, Forester Court, Wis., 53209), the athletes completed a discontinuous incremental test involving 3 min work efforts, each 1 km·hr¹ faster than the previous stage, separated by a 30 second rest period to allow for the measurement of blood lactate, and rate of perceived exertion (RPE; Borg, 1970). Each athlete completed between 5 and 9 submaximal stages, starting at an intensity below lactate threshold (typical male speed: 14 km·hr⁻¹; female speed: 11 km·hr⁻¹; Blood lactate was checked after the warm up, and the starting speed of the incremental test was reduced if lactate was above 2 mMol·L⁻¹). The warm up was conducted at the same speed as the first 3 min stage of the incremental test. The incremental test was terminated once blood lactate exceeded 4 mMol·L⁻¹. Prior to the submaximal test, the athletes were fitted with a mask for breath-by-breath expired air analysis (Jaeger Oxycon Pro, Hoechberg, Germany) and a heart rate (HR) monitor. HR was measured continuously and recorded throughout the exercise protocol (Polar Team System®, Polar U.K.). Following completion of the submaximal exercise test, athletes were given a 5 min rest period, whereby additional redox measures were immediately taken, before undergoing the maximal progressive exercise test to exhaustion at a constant speed, 2 km·hr¹ slower than the final speed of the sub-maximal test. The test began at a 1% gradient and increased by 1% every minute until volitional exhaustion. Heart rate peak (HR_{PEAK}) was the highest HR value derived by Polar ProTrainer 5® software set at a 5 second sampling rate. Maximal aerobic capacity (\dot{V}O₂max) was defined as the highest 30 second average during the maximal exercise test.

Height and body mass were recorded and skinfold thickness (mm) measured by the same researcher, 7 sites were measured for the calculation of body fat (%) using the equation of Jackson & Pollock, 1978. Br. J. Nutr. 40: 497-504. The researcher was accredited through the International Society for the Advancement of Kinanthropometry (ISAK).

Blood sampling. Capillary blood samples were obtained at rest from the earlobe at the following time points: baseline (pre-exercise), immediately post sub-maximal exercise, immediately post-maximal exercise, and after recovery from the maximal exercise test (static recovery, 20 minutes post maximal test, supine).

Athletes were allowed access to sips of water post-exercise and into the recovery period should they complain of a dry mouth and thirst. Pre- and post-exercise plasma volume (PV) changes were estimated via the determination of hematocrit (Hct) and hemoglobin (Hb) concentration, using the formula of Dill & Costill, 1974. Journal of Applied Physiology 37: 247-248.

Whole blood capillary samples, 50 μL for FORD, and 20 μL for FORT were sampled from the ear lobe in heparinized capillary tubes. These were immediately mixed with reagent and centrifuged at 5,000 rpm for 1 minute, and analysed according to the manufacturers instructions using a Callegari analyzer (Callegari SpA, Catellani Group, Parma, Italy) controlled at 37° C. with absorbance set at a wavelength of 505 nm for the calculation FORT and FORD. Methodology for the FORT and FORD assay was performed as described previously in paragraphs [0036]-[0042]. Intra- and inter-assay coefficients of variation (CV) for FORT and FORD were <5% and 7% respectively.

Hematocrit was determined by capillary collection using 60 μL sodium heparinised tubes, then centrifuged at 3000 rpm for 3 min. The packed cell volume was measured using a micro-haematocrit reader (Hawksley, UK). A 10 μL blood sample was collected in a Hemocue™ 201+ microcuvette and analysed in a Hemocue™ 201+(AB Leo Diagnostics, Helsinborg, Sweden) dual wavelength photometer for haemoglobin readings.

Capillary blood samples were also obtained following every phase of exercise and immediately analysed for blood lactate using a Biosen C-Line analyzer (EFK Diagnostic, Barleben, Germany). These were used to identify the running speed corresponding to the lactate threshold (LT; defined as the first rise in blood lactate exceeding 0.4 mM), and the running speed corresponding to 3 mM (vLTP). The Orreco Lactate-OR web based application was used to define these points (Newell, et al., 2014. Journal of Sports Sciences: 1-2).

Statistical analysis. All statistics were carried out using Minitab Inc. version 16. (USA). The distributions of all variables were assessed for normality with box-plots, and calculated using the Anderson-Darling test. Following normality tests, a general linear model (GLM) was used to test for an effect of exercise (5 levels: rest, warm-up, sub-max, maximal exercise and recovery) on FFT measures, and for an effect for gender (2 levels). In addition, a GLM was used to test for differences between short distance events (400 m, 800 m, 1500 m) and long distance athletes (5 k, 10 k, marathon, triathlon) for FORT and FORD responses to exercise (rest, warm-up, sub-max, maximal exercise and recovery). If a significant interaction was evident, then pairwise comparisons were performed using the Tukey post hoc test. All FFT measures at rest and exercise data were analysed both with and without adjusting for PV to assess the need to control for changes in PV in relation to exercise and redox measures. Cohen's d effect sizes (d) were then used to calculate the magnitude of the standardised difference in means where significant, and reported as 0.2 (small), 0.5 (moderate), 0.8 (large), and 1.3 (very large). Significant relationships between variables were explored, for gender, and then combined male and female FORT, FORD, age and exercise intensity variables using Pearson's correlation coefficients. Data is presented as mean±SD with significance accepted at p<0.05.

Results. Subject characteristics and physiological variables are presented in Table 5, which shows significant differences between male and female athletes for speeds at lactate threshold (p=0.05, d=1.03), and lactate turn point (p=0.03, d=1.22) and velocity at \dot{V}O₂ₘₐₓ(v\dot{V}O₂ₘₐₓ) (p=0.02, d=1.36) (Table 5).

TABLE 5
Athlete characteristics

| | Female athletes | Male athletes |
|---|---|---|
| Variable | (n = 7) | (n = 15) |
| Age (y) | 27.9 ± 5.3 | 30.7 ± 9.1 |
| Weight (kg) | 59.8 ± 5.9 | 68.8 ± 6.1 |
| Height (cm) | 170.9 ± 6.4 | 178.2 ± 5.2 |
| Body fat (%) | 10.6 ± 3.2 | 7.7 ± 3.4 |
| Sum Σ7 skinfolds (mm) | 59.2 ± 19.7 | 43.6 ± 19.0 |
| Lactate threshold (km · h⁻¹) | 13.0 ± 1.2 | 14.9 ± 2.3* |
| Lactate turnpoint (km · h⁻¹) | 15.1 ± 1.2 | 17.2 ± 2.1* |
| v\dot{V}O₂ₘₐₓ (km · min⁻¹) | 17.2 ± 1.4 | 19.4 ± 1.8* |
| \dot{V}O₂ₘₐₓ (ml · kg⁻¹ · min⁻¹) | 61.4 ± 7.3 | 68.7 ± 5.8 |
| \dot{V}O₂ₘₐₓ range (ml · kg⁻¹ · min⁻¹) | 53-71 | 67-80 |

*p < 0.05

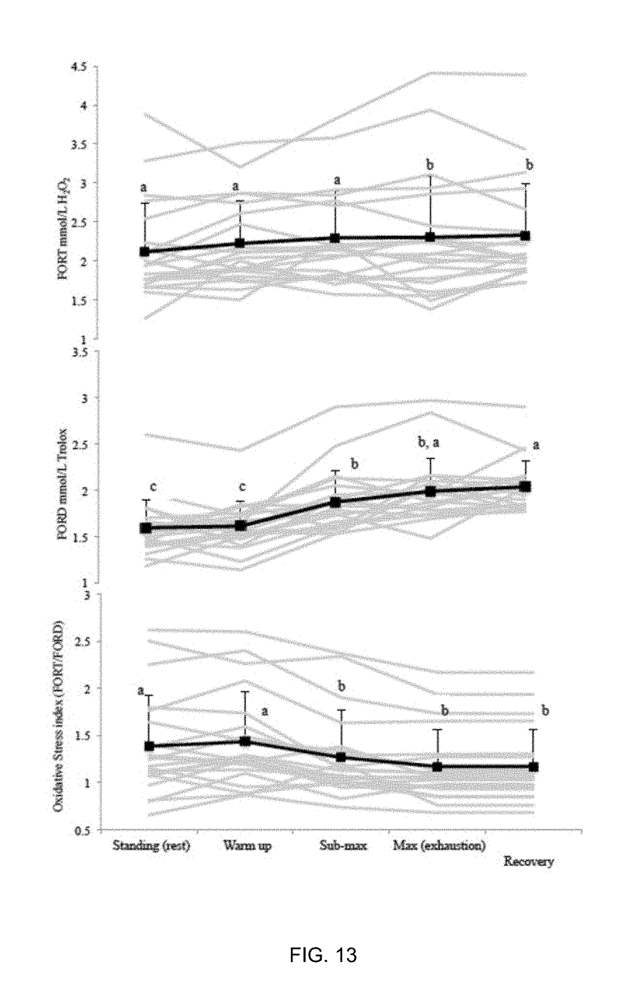

FORD and FORT. There were no effects for gender on plasma FORD (p=0.48) and FORT (p=0.42); thus male and female data were combined. The combined results adjusted for PV at rest, and after warm up, sub-maximal exercise, maximal exercise and recovery are presented in FIG. 13. The importance of correcting for PV changes with exercise was evident, because un-adjusted PV FORD and FORT concentrations resulted in additional interactions not otherwise present. Significant relationships between the redox and physiological variables are reported in Table 6.

TABLE 6
Correlation matrix for FORD and physiological
variables (LT, LTP and V\dot{O}₂ₘₐₓ)

| | FORD | FORD | FORD | | | |
|---|---|---|---|---|---|---|
| | rest | max | recovery | \dot{V}O₂ₘₐₓ | LTP | LT |
| FORD at | | | .526* | −0.093 | .443 | .480* |
| rest | | | .014 | .688 | .051 | .028 |
| FORD | .742** | | .658** | .162 | .404 | .444* |
| maximal | .000 | | .001 | .483 | .078 | .044 |
| FORD | .526* | .658** | | .457* | .517* | .489* |
| recovery | .014 | .001 | | .037 | .019 | .024 |
| \dot{V}O₂ₘₐₓ | −0.093 | .162 | .457* | | .219 | .253 |
| | .688 | .483 | .037 | | .355 | .269 |
| LTP | .443 | .404 | .517* | .219 | | .975** |
| | .051 | .078 | .019 | .355 | | .000 |
| LT | .480* | .444* | .489* | .253 | .975** | |
| | .028 | .044 | .024 | .269 | .000 | |

*p < 0.05
**p < 0.01

Oxidative Stress Index (OSi). In particular embodiments, the OSi refers to the ratio of FORT to FORD and provides a basic indication of the pro-anti-oxidant balance in plasma. An interaction was evident for the OSi with time (p<0.001), with no effect observed for gender (p=0.35); see FIG. 13.

There were no significant differences among short distance events (400 m, 800 m, 1500 m) or among long distance athletes (5 k, 10 k, marathon, triathlon) for FORT and FORD across any of the time points measured (p>0.05).

Discussion Significant ARH in elite male and female endurance athletes in response to sub-maximal and maximal exercise is reported, with no differences between genders. Using a clinical redox POC test to assess ARH, significant increases in both FORD and FORT were evident, with the ratio of the two measures indicating an overall reduction in OS in response to exercise. The increase in FORD was greater than the increase in FORT. Furthermore, a significant relationship between plasma FORD both at rest, and at intensities reflective of the athletes aerobic conditioning was observed. Thus aerobic fitness influences plasma FORD in elite athletes.

This suggests that a higher level of aerobic conditioning is related to the plasma FORD response to exercise. This might be explained on the basis that the more highly trained the athlete (i.e. those attaining a higher velocity at LT, LTP and \dot{V}O₂ₘₐₓ) the greater their resting GSH concentrations, and capacity for the mobilisation of endogenous anti-oxidant enzymes (i.e. GSH) into the blood in response to maximal exercise, to combat increases in RNOS.

The rise in plasma FORD after exercise may be accounted for by the rise in plasma ascorbic acid (vitamin C), released from the adrenal glands into the circulation which occurs with exercise. Plasma vitamin C increases in response to stress hormones (Padayatty, et al., 2007. Am. J. Clin. Nutr. 86: 145-149), which increase with exercise duration and intensity, and the plasma vitamin C concentration is reported to contribute to the anti-oxidant activity of the FORD assay (Palmieri & Sblendorio, 2007. Eur Rev Med Pharmacol Sci 11: 309-342). Another important factor contributing to the rise in plasma FORD is the tri-peptide glutathione (GSH; Palmieri & Sblendorio, 2007. Eur Rev Med Pharmacol Sci 11: 309-342). Several studies in elite athletes have reported acute and chronic changes in blood GSH in response to exercise (Lewis, et al., 2015. Sports Med 45: 379-409), with exercise training known to elicit the up-regulation of intra-cellular GSH (Elokda & Nielsen, 2007. European Journal of Cardiovascular Prevention & Rehabilitation 14: 630-637), and intra-cellular GSH being a significant source of blood GSH Giustarini, et al., 2008. Blood Cells, Molecules, and Diseases 40: 174-179). Thus plasma changes in the athletes FORD values may largely reflect changes in vitamin C and GSH; this was not tested experimentally in the present study. However, a significant relationship between resting FORD and red blood cell GSH in well-trained athletes has been reported (Lewis, et al., 2016. PLoS ONE 11: e0149927), with 15-20% of RBC GSH exported into plasma on a daily basis Giustarini, et al., 2008. Blood Cells, Molecules, and Diseases 40: 174-179).

Other large and small molecular weight molecules reported to make a significant contribution to the anti-oxidant capacity of blood include, ceruloplasmin, albumin, bilirubin, and melatonin. (Atanasiu, et al., 1998. Molecular and cellular biochemistry, 189: 127-135; Benitez, et al., 2002. Atherosclerosis, 160:223-232; Benot, et al., 1999. Journal of pineal research, 27: 59-64).

There is evidence that melatonin might have contributed to the observed acute rise in blood anti-oxidant capacity (i.e. FORD) with exercise. Melatonin is a potent anti-oxidant, and is known to increase acutely with heavy exercise performed in natural daylight hours (Atkinson, et al., 2003. Sports Medicine, 33: 809-831). Furthermore, the diurnal variation in blood anti-oxidant capacity reflects the changes in melatonin, with maximal nocturnal values reported for both. Finally, using bright light to blunt the nocturnal rise in melatonin prevents the accompanying rise in serum anti-oxidant capacity (Benot, et al., 1999. Journal of pineal research, 27: 59-64). The contribution of various serum proteins, bilirubin and melatonin to the anti-oxidant capacity of the FORD assay warrants investigation.

The athletes recruited in the current study did not report fatigue or performance concerns on interview, and a significant rise in FORD following exercise was observed, thus supporting the notion that the athletes were not fatigued excessively at the time of the study. Therefore, this study provides reference data for which the responses in fatigued or under-recovered athletes may be compared against.

Increases in FORT occurred only with maximal exercise, remaining elevated following 20 minutes recovery. There were no differences between rest and warm up and submaximal exercise. Therefore, it appears that unless the athlete's physiological systems become acutely stressed when exercising to exhaustion, there may be sufficient reserve with the endogenous anti-oxidant enzymatic systems to combat any exercise induced increase in RNOS, in the healthy elite athlete.

The plasma FORD increase in response to exercise was greater than the FORT response, reflecting a mobilization of anti-oxidants. It is well documented that endurance training increases anti-oxidant enzyme activity in blood, red blood cells and skeletal muscle, with elite athletes having well adapted anti-oxidant enzymatic systems leading to a reduced ARH and OS increasing across the season (Lewis, et al., 2015. Sports Med 45: 379-409). Indeed, some of the largest reported changes in redox homeostasis occurred in the general preparation phases of periodized training programs, such as when athletes are returning to training after a transition period of minimal training and detraining (Kyparos, et al., 2009. J Strength Cond Res 23: 1418-1426; Kyparos, et al., 2011. Eur J Appl Physiol 112: 2073-2083).

In studies examining redox responses to exercise consideration needs to be given to the timing of the post-exercise sample. Given that elite athletes were assessed, a sampling point immediately post-maximal exercise and 20 minutes into recovery was selected, as used by other studies in elite athletes (Palazzetti, et al., 2003. Can J Appl Physiol 28: 588-604; Tanskanen, et al., 2010. Journal of Sports Sciences 28: 309-317).

PV changes were controlled for in the study and the analysis was performed with and without adjustments. It has been reported that failure to control for PV changes may in part account for some of the discrepancies reported in the literature in studies of exercise and OS (Farney, et al., 2012. Medicine & Science in Sports & Exercise 44: 1855-1863). Indeed, adjusting for PV influenced the results for both the FORD and FORT, and thus a failure to control for PV in exercise studies may influence the outcome and increase the chances of significant findings (type 1 error) in relation to pre- vs. post-exercise observations for ARH.

Finally, the data from endurance athletes, and from those who run short vs. long distances was pooled. In fact, there were no significant differences among short distance events (400 m, 800 m, 1500 m) or among long distance athletes (5 k, 10 k, marathon, triathlon) for FORT and FORD across any of the time points measured.

### Example 5

Case Studies. Case Study 1. Soccer player. The athlete reported relatively low energy levels and muscle aches on the day of testing and rates muscle soreness as 2 out of 4 (1 is the worst). This athlete played a lot and was a regular scorer. The athlete's FORT measured 2.47, i.e. above the athlete's critical threshold and the FORD measured 1.36. There was also out of range superoxide dismutase (SOD). Creatine kinase (CK) was very high suggesting a high amount of muscle damage. The case study 1 athlete experienced a strained hamstring 5 days later.

Case Study 2. Soccer player. The athlete reported moderate energy levels. At the time, the athlete was a new player with an increased workload. The player was physically struggling. The athlete's FORT measured 2.68, i.e. above the athlete's critical threshold, and the FORD measured 1.22. There was also out of range superoxide dismutase (SOD) and Glutathione (GSH). Urea was also increased—possibly suggesting increased protein breakdown. The case study 2 athlete experienced a tweaked hamstring in training immediately after testing.

Case Study 3. Runner. The athlete reported a sore throat/blocked nose/cough on the day of testing. The athlete also exhibited fever/temperature symptoms on the day of testing. Historically, this athlete did not tend to communicate wellness with coaching staff and the coach. FORT value was significantly higher than the previous two readings (still establishing a baseline). This result was flagged to the coach and training was modified. A likely injury was averted.

Case Study 4. Basketball player. The athlete reported relatively low energy levels. The athlete was in the midst of career best start to the season and was a key player with a high game load. FORT value was above critical difference threshold and FORD value was below the critical difference threshold. The player picked up a soft tissue tear 2 days later and was predicted to be sidelined for 6 weeks.

Case Study 5. Pre-infection (incubation period). The athlete reported increased fatigue, being tired, and “not feeling themselves.” The athlete had returned from long travel (i.e. 4 hour flight, and a 10 hour road trip behind the wheel). The athlete was asymptomatic for infection at this time (other than reporting fatigue). FORT value was above the critical difference threshold, and the athlete came down with a symptomatic upper respiratory tract infection 36 hours later. The athlete was removed from the training environment for 72 hours and training intensity and volume was reduced until FORT recovered. Zinc lozenges were also prescribed.

Case Study 6. Upper Respiratory Tract Infection. The athlete reported increased fatigue, a sore painful throat, cough, and generally feeling unwell. The athlete presented with clear signs of an infection to the upper respiratory tract. FORT value was above the critical difference threshold. The athlete was prescribed a period of low intensity training until the resolution of symptoms. FORT values recovered post infection. The athlete was instructed to monitor symptoms and not increase intensity until clear improvement was evident.

Case Study 7. Gastro-intestinal Infection. The athlete reported increased fatigue, diarrhea, stomach cramps, and generally feeling unwell. The athlete had returned from a competition in Cairo, Egypt where the athlete had eaten unfamiliar foods. FORT value was above the critical difference threshold. The athlete needed a course of antibiotics. FORT value recovered with resolution of the infection. The athlete was removed from the training environment until the diarrhea disappeared. Dietary advice included probiotics and discontinuance of dairy foods, and reduced fibre intake until symptoms abated.

Case Study 8. Urinary Tract Infection. The athlete reported increased fatigue and generally feeling unwell. The athlete complained of fatigue and tiredness that had been present for a period of 10 days. FORT value above the critical difference threshold. The athlete was diagnosed with urinary tract infection on medical examination and tests and was given a course of antibiotics. Training was modified given the fatigue and infection, and the course of antibiotics was taken as prescribed.

Case Study 9. Use of Oral Contraceptive Pill (OCP). The athlete reported use of an OCP in an attempt to control cycle length and mood state. The athlete was very concerned her menstrual cycle would land on key competition days. FORT values gradually crept up, until after 4-5 weeks the values were above her threshold. The athlete did not perform to full capability (although this was multi factorial). Strategies aimed at enhancing recovery and reducing oxidative damage were implemented in an attempt to attenuate the effects of the OCP e.g. use of polyphenol supplements.

Case Study 10. Bone fracture (trauma, acute injury). The athlete reported being struck on the arm whilst fencing, resulting in an acute fracture of the bones residing in the hand. The athlete presented with pain, swelling, redness in the hand; all the signs of acute inflammation. FORT values recorded 48 hours after injury exceeded the athlete's threshold (values were not recorded at the time of injury as the athlete was overseas). The athlete rested, and refrained from training until the inflammation subsided, and then returned to light training. FORT values recovered as the injury healed, and tracked the course of recovery (3 measures were taken over 3 weeks; one every 7 days)

Case Study 11. Bone stress (overuse injury)—retrospective analysis of data. The athlete reported painful shins, and was placed in a boot by the medical team following a diagnosis of lower limb bone stress. FORT values taken at the time of the injury exceeded the athlete's threshold. The athlete was in a boot for several weeks, and missed a total of 8 weeks of full training. FORT values recovered with resolution of the injury and return of “normal training”. The athlete finished the season with medical management of the injury.

Case Study 12. Dislocated shoulder (acute injury). The athlete reported falling whilst cross country running at home, resulting in a dislocated shoulder. The athlete presented a week post the injury in the sports clinic once back at the training center. FORT values were above threshold. The athlete was put on a period of restricted training. FORT values recovered with resolution of the injury and return to training. Strategies aimed at enhancing recovery were implemented

Case Study 13. Non-functional overreaching. The athlete reported considerable fatigue and was unable to undertake any training. The athlete had just returned (3 days prior) from three weeks of intensified training at altitude. Furthermore, prior to testing that morning, the athlete had spent 2 days at a festival, drinking, and getting very little sleep, rest or quality nutrition. The athlete appeared emotional at the time of testing. FORT values were substantially above the critical difference threshold. The athlete was given 3 days of compete rest, and then one week of light aerobic training only. FORT values decreased rapidly as the fatigue resolved. Strategies aimed at enhancing recovery and reducing oxidative damage were implemented in an attempt to accelerate recovery.

Case Study 14. Bout of gross under performance. The athlete reported an infection 2-3 weeks prior to performing at a major championships. The symptoms had resolved, 1 week prior to the competition, and the athlete felt better. FORT values were above the threshold with the infection, and although had started to decline with resolution of the infection, still remained slightly above the threshold at the time of the competition. There was poor physical performance at the competition, resulting in the athlete's worst performance at a major swim competition in several years.

Case Study 15. Period of hypobaric hypoxic aerobic training (altitude). The athlete reported several weeks of training at an altitude camp. The athlete was healthy, eating, and training well. FORT values gradually declined across the training period. FORT results suggest/support that the training period enhanced anti-oxidant “defenses” i.e. endogenous anti-oxidant enzymes. The changes in FORT values suggested up-regulation of the athlete's endogenous anti-oxidant enzymes and thus a useful marker of adaptation to the training program and environment (i.e. lower FORT values as a result of the athlete being fitter aerobically)

Case Study 16. Summary of ongoing case study. Male basketball player age 38 years (relatively old by NBA standards), suffering episodes of achilles tendinopathy (tedinopathy is often ongoing in the background with athletes and has to be managed) and a sustained level of oxidative stress (pro-oxidant score repeatedly >2.1 and anti-oxidant score around 1.0, occasionally lower). The player was rested with continuous rehabilitation work and a full dietary review was undertaken (in particular protein intake was increased, and this is an important precursor to the ‘master anti-oxidant’ glutathione). The player has gradually been introduced back into the game as the tendinopathy has recovered and is now playing full minutes. Another factor with this player which is likely to have contributed to his poor redox status is sleep deprivation caused by being a recently new parent.

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” As used herein, the transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. As used herein, a material effect would cause a statistically-significant reduction in ability to detect injury or illness in a subject.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, if references have been made to patents, printed publications, journal articles and other written text throughout this specification (referenced materials herein), each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Ed. Anthony Smith, Oxford University Press, Oxford, 2004).

1.-32. (canceled)

33. A method of reducing risk of at least one of injury or unexplained underperformance syndrome (UUPS) in a group of individuals, the method comprising:
obtaining contextual data from each individual in the group of individuals;
obtaining a plurality of physiological samples from each individual in the group of individuals, wherein at least two samples for each individual included in the group of individuals are obtained at least a week apart;
detecting, in at least a portion of the plurality of physiological samples, first values of one or more pro-oxidant markers and one or more second values of one or more anti-oxidant markers using at least two colorimetric assays;
calculating a critical difference threshold for each individual of the group of individuals;
obtaining additional physiological samples for an individual of the group of individuals;
detecting first additional values of the one or more pro-oxidant markers and second additional values of the one or more anti-oxidant markers in the additional physiological samples using the at least two colorimetric assays;
determining that at least one of the first additional values or the second additional values are outside of the critical difference threshold of the individual;
identifying the individual as being at least one of at increased risk for injury or at increased risk for UUPS based at least partly on the at least one of the first additional values or the second additional values being outside of the critical difference threshold of the individual; and
determining a recommendation for an intervention to reduce risk of at least one of injury or UUPS for the individual.

34. The method of claim 33, wherein calculating the critical difference threshold for the individual includes:
assaying at least four physiological samples of the individual to obtain free oxygen radical test (FORT) values and free oxygen radical defense test (FORD) values;
calculating an upper threshold for the FORT values;
calculating a lower threshold for FORD values; and
determining a difference between the upper threshold and the lower threshold to establish the critical difference threshold.

35. The method of claim 33, wherein the contextual data indicates one or more of dietary habits, rest habits, mood, energy levels, or injury status of individuals included in the group of individuals.

36. The method of claim 33, further comprising:
providing at least one of the first values of the one or more pro-oxidant markers or the one or more second values of the one or more anti-oxidant values to a system for analysis.

37. The method of claim 33, further comprising:
providing the additional physiological samples to a redox analyzer to detect the first additional values of the one or more pro-oxidant markers and the second additional values of the one or more anti-oxidant markers.

38. The method of claim 33, further comprising:
generating a graphical user interface that indicates the critical difference threshold over a period of time and that indicates values of the one or more pro-oxidant markers and values of the one or more anti-oxidant markers over the period of time.

39. The method of claim 38, wherein the graphical user interface includes a user interface element indicating whether at least one of the first additional values of the one or more pro-oxidant markers or the second additional values of the one or more anti-oxidant markers are outside of the critical difference threshold at a specified time.

40. The method of claim 33 further comprising:
modifying the critical difference threshold to produce a modified critical difference threshold based at least partly on the first additional values of the one or more pro-oxidant markers and the second additional values of the one or more anti-oxidant markers.

41. The method of claim 33, wherein the recommendation is displayed in conjunction with an application executing on a computing device.

42. A method comprising:
obtaining a plurality of physiological samples from an individual;
detecting first values of one or more pro-oxidant markers in the plurality of physiological samples using at least a first colorimetric assay;
detecting second values of one or more anti-oxidant markers in the plurality of physiological samples using the at least a second colorimetric assay;
calculating a critical difference threshold for the individual based at least partly on the first values of the one or more pro-oxidant markers and the second values of the one or more anti-oxidant markers, the critical difference threshold including an upper threshold corresponding to values of the one or more pro-oxidant markers and a lower threshold corresponding to values of the one or more anti-oxidant markers;
detecting first additional values of the one or more pro-oxidant markers and second additional values of the one or more anti-oxidant markers in additional physiological samples of the individual using at least the first colorimetric assay and the second colorimetric assay;
analyzing the first additional values and the second additional values in relation to the critical difference threshold; and
generating a graphical user interface indicating the first additional values and the second additional values in relation to the critical difference threshold.

43. The method of claim 42, further comprising:
determining that the individual is at an increased risk of at least one of injury, illness, or unexplained underperformance syndrome (UUPS) based at least partly on at least one of the first additional values or the second additional values being outside of the critical difference threshold; and
generating a recommendation for an intervention to reduce a risk of the individual in regard to the at least one of injury, illness, or UUPS.

44. The method of claim 42, further comprising:
determining that the individual is well based at least partly on at least one of the first additional values or the second additional values being within the critical difference threshold; and
generating a recommendation to maintain a wellness of the individual.

45. The method of claim 42, wherein the plurality of physiological samples and the additional physiological samples of the individual are obtained from at least one of blood, saliva, urine, tear, deoxyribonucleic acid (DNA), perspiration, or extracellular fluid.

46. The method of claim 42, wherein:
the one or more pro-oxidant markers are obtained using a free oxygen radical test (FORT) assay and the one or more anti-oxidant markers are obtained using a free oxygen radical defense test (FORD) assay; and
the individual is an athlete, physical therapy patient, member of a corporate wellness program, or a competing animal.

47. A system, comprising:
a redox analyzer; and
a computing device having:
one or more processors; and
memory coupled to the one or more processors, the memory storing instructions that, when executed, cause the one or more processors to perform operations comprising:
receiving, from the redox analyzer, sample values associated with a physiological sample from an individual, the sample values indicating pro-oxidant values and anti-oxidant values associated with the physiological sample;
retrieving, from a database, historical values associated with historical physiological samples;
determining, based at least in part upon the historical values, a critical difference threshold for the individual;
comparing the sample values with the critical difference threshold to determine that one or more of the sample values are outside a range defined by the critical difference threshold;
detecting, based at least in part upon the one or more of the sample values being outside the range, that the individual is at increased risk of at least one of injury or unexplained underperformance syndrome (UUPS);
generating an alert indicating that the individual is at increased risk of at least one of injury or UUPS; and
presenting, on a display device associated with the computing device, an indication of the alert and a recommended action plan.

48. The system of claim 47, wherein the redox analyzer and the computing device are contained within a common housing.

49. The system of claim 47, wherein the operations further comprise:
updating the database with the sample values; and
determining, based at least in part upon the historical values and the sample values, a new critical difference threshold.

50. The system of claim 47, wherein the recommended action plan includes modifying one or more of a diet, an exercise regimen, an exercise intensity, or a sleep pattern of the individual.

51. The system of claim 47, wherein:
the critical difference threshold is determined, at least in part, on a Bayesian predictive model; and
the redox analyzer provides at least one free oxygen radical test (FORT) value and at least one free oxygen radical defense test (FORD) value.

52. The system of claim 47, wherein the operations further comprise presenting, on the display device, a visual indication of the critical difference threshold and the sample values.
